(19) United States
(12) Patent Application Publication
(10) Pub. No.: US 2020/0138977 A1
(43) Pub. Date: May 7, 2020

(54) NUCLEIC ACID-CATIONIC POLYMER COMPOSITIONS AND METHODS OF MAKING AND USING THE SAME

(71) Applicant: Yissum Research Development Company of the Hebrew University of Jerusalem Ltd., Jerusalem (IL)

(72) Inventors: Abraham HOCHBERG, Nes Ziona (IL); Jennifer GALLULA, Jerusalem (IL)

(73) Assignee: Yissum Research Development Company of the Hebrew University of Jerusalem Ltd., Jerusalem (IL)

(21) Appl. No.: 16/732,770

(22) Filed: Jan. 2, 2020

Related U.S. Application Data
(63) Continuation of application No. 15/571,699, filed on Nov. 3, 2017.

(30) Foreign Application Priority Data
May 5, 2015 (IL) 238643

(51) Int. Cl. A61K 48/00; A61K 9/00; A61K 9/19; A61P 35/00; C12N 15/88

(52) U.S. Cl. CPC A61K 48/0041; A61K 9/0019; A61K 9/19; A61K 48/0091; Y02A 50/471; A61K 48/0066; A61P 35/00; C12N 15/88; A61K 48/0058

(57) ABSTRACT

Provided herein are pre-lyophilized compositions containing a nucleic acid, a cationic polymer, and a carbohydrate. Also provided are lyophilized compositions of matter and reconstituted compositions as well as methods of making using the same for treating tumors in patients.

### TECHNOLOGICAL FIELD

The invention relates generally to the fields of tumor cell biology, gene therapy, and cancer treatment. More specifically, the invention relates to compositions of matter of nucleic acids and cationic polymers that can be lyophilized as well as to methods of making and using the same.

### BACKGROUND

The use of DNA plasmids as drugs to treat diseases by therapeutic delivery into a patient's cells is one of the upcoming technologies in the development of novel drug agents for a wide spectrum of pathologies that to date have been considered untreatable.

In order to achieve an effective delivery of a functional DNA plasmid inside cells by overcoming the nuclease degradation within the body, the nucleic acid molecule should be packaged within a “vector”.

Although initially most research on gene therapy focused on the development of viral-mediated vectors, non-viral transfectants have been developed as potentially safe and effective gene therapy delivery methods.

Compared to viral vectors, these non-viral delivery systems demonstrate several advantages, including low toxicity and immunogenicity, resistance to nuclease, and improved safety profiles.

Non-limiting examples of such non-viral delivery systems include new molecules such as lipoplexes and/or polyplexes that have been created and are able to protect the DNA from degradation during the transfection process.

To form lipoplexes, plasmid DNA is covered with cationic lipids having an organized structure (e.g., micelles or liposomes). These cationic lipids complex with negatively charged DNA, and the positively charged lipids also interact with the cell membrane, thereby allowing endocytosis of the lipoplex to occur. The DNA within the lipoplex is subsequently released into the cytoplasm.

Polyplexes are complexes of DNA with polymers. Typically, polyplexes utilize cationic polymers, which interact and complex with the polyanionic DNA.

Among the wide range of non-viral vectors that have been developed, the cationic polymer linear polyethylenamine (PEI) (see U.S. Pat. No. 6,013,240, which is herein incorporated by reference in its entirety) has shown several advantages in plasmid delivery (see Pathak et al., Biotechnol. J. 4:1559-1572 (2009); Kasper et al. Journal of Controlled Release 151:246-255 (2011)) and has been used in clinical studies (see Gofrit et al., The Journal of Urology 191(6):1697-1702 (2014); Abraham et al., The Journal of Urology 180(6):2379-2383 (2008)). Thus, PEI is considered as the “golden standard” of the non-viral vectors. (See Pathak et al., Biotechnol. J. 4:1559-1572 (2009); Kasper et al. Journal of Controlled Release 151:246-255(2011)).

Polyplexes based on Linear Polyethylenimine (LPEI) and DNA plasmids, are known for their advantageous qualities with regard to transfection efficiency over a wide range of transfectants. However the stability of such complexes in aqueous solutions is limited and the need for freshly prepared complexes prior to administration increase the risk of batch-to-batch variations, especially in high DNA concentration solutions. Under such conditions, the reproducibility and the controlled quality of those complexes cannot be guaranteed to the extent required in pharmaceutical products.

### BACKGROUND ART

[1] U.S. Pat. No. 6,013,240
[2] Pathak et al., Biotechnol. J. 4:1559-1572 (2009)
[3] Kasper et al. Journal of Controlled Release 151:246-255 (2011)
[4] Gofrit et al., The Journal of Urology 191(6):1697-1702 (2014)
[5] Abraham et al., The Journal of Urology 180(6):2379-2383 (2008)
[6] Pathak et al., Biotechnol. J. 4:1559-1572 (2009)
[7] PCT/IL1998/000486 (WO 1999/018195)
[8] PCT/IL2008/001405 (WO 2009/053982)
[9] PCT/IL2006/001110 (WO 2007/034487)
[10] PCT/IL2006/000785 (U.S. Pat. No. 8,067,573)
[11] PCT/IL2008/000071 (U.S. Pat. No. 7,928,083)
[12] Brus et al., Journal of Controlled Release 95:119-131 (2004)
[13] Kasper et al., European Journal of Pharmaceutics and Biopharmaceutics 77:182-185(2011)
[14] Julia Christina Kasper, Doctoral Thesis “Lyophilization of Nucleic Acid Nanoparticles—Formulation Development, Stabilization Mechanisms, and Process Monitoring” (2012)

### SUMMARY OF THE INVENTION

Thus, a need remains in the art for methods of preparing nucleic acid-cationic polymer compositions of matter that are more streamlined and that can be easily scaled to industrially useful proportions.

The inventors of the invention disclosed herein have developed a unique process for the production of pre-lyophilized, lyophilized and reconstituted composition/formulation containing a nucleic acid, a cationic polymer and a carbohydrate, which stability in aqueous solutions is far improved, providing an excellent replacement to similar compositions known in the art. The processes of the invention, as well as the compositions produced thereby, provide an answer to the need for accurately prepared, safe and industrially scalable complexes for therapeutic applications.

The processes of the invention permit industrial manufacture of stable, accurately dosable and homogenous composition/formulations which may be formulated into a lyophilized or pre-lyophilized formulation without negatively affecting the constitution, integrity, stability and biological availability of any of the components of the formulation.

Thus, in a first aspect, the invention provides a process for the preparation of a composition comprising at least one nucleic acid, at least one cationic polymer, and at least one carbohydrate the process comprising adding a nucleic acid/carbohydrate solution into a cationic polymer/carbohydrate solution, under conditions permitting formation of a complex between the at least one nucleic acid and the at least one cationic polymer.

In some embodiments, the nucleic acid/carbohydrate solution and the cationic polymer/carbohydrate solution may be each separately and independently prepared. Each of the solutions may be prepared well in advance of their combination, as recited, at the same time or at different points in time.

In some embodiments, the process comprises obtaining each of the two solutions and adding the nucleic acid/carbohydrate solution into the cationic polymer/carbohydrate solution, and not vice versa, under conditions permitting formation of a complex between the at least one nucleic acid and the at least one cationic polymer, to obtain the composition of the invention.

In some embodiments, the process comprises:

(a) obtaining a solution of at least one nucleic acid and at least one carbohydrate;

(b) obtaining a solution of at least one cationic polymer and at least one carbohydrate;

(c) adding the nucleic acid/carbohydrate solution into the cationic polymer/carbohydrate solution, under conditions permitting formation of a complex between the at least one nucleic acid and the at least one cationic polymer; and

(d) optionally lyophilizing the combined nucleic acid/cationic polymer/carbohydrate solution to form the lyophilized composition.

In some embodiments, the process comprises a step of making the nucleic acid/carbohydrate solution and a separate step for making the cationic polymer/carbohydrate solution.

In some embodiments, the process thus comprises:

(a) mixing an amount of the at least one nucleic acid with a first amount of the at least one carbohydrate to form a nucleic acid/carbohydrate solution; and

(b) mixing an amount of the at least one cationic polymer with a second amount of the at least one carbohydrate to form a cationic polymer/carbohydrate solution.

As noted above, the at least one carbohydrate is used in methods of the invention in separate hatches or quantities. A first batch or quantity, or first amount is mixed with the at least one nucleic acid, and a second batch or quantity, or second amount is mixed with the at least one cationic polymer. The first or second amounts are determined and selected to be the minimum amount of the at least one carbohydrate sufficient to permit formation of the complex and provide a stable, optionally solid, product.

As used herein, the composition or formulation of the invention comprises a complex between the at least one nucleic acid and the at least one cationic polymer, to which the stability and uniqueness of the composition is attributed. The term “complex”, “polyplex”, “polyplex formulation”, “polyplex composition of matter”, “composition of matter”, and the like are used interchangeably herein to refer to the compositions/formulations of the invention, as a whole and not to any particular component thereof.

As exemplified herein, each of the solutions of steps (a) and (b) may be prepared independently of the other and may be stored before use. Each of the solutions may be prepared in sequence, as recited above, or in any other sequence, provided that they are added to each other as indicated in step (c), namely adding the nucleic acid/carbohydrate solution into the cationic polymer/carbohydrate solution, and not vice versa. This particular order-specific addition of one of the solutions into the other, permits facile formation of a unique and stable complex between the at least one nucleic acid and the at least one cationic polymer; a complex which cannot be formed in large quantities when the solutions are added in a reverse way.

This addition of one of the composition components into the other and not vice versa, as recited, permits also reducing the amount of the carbohydrate material and subsequently increasing the relative amount of the at least one nucleic acid in the composition. In other words, by being able to reduce the amount of the at least one carbohydrate, while keeping the amount of the at least one nucleic acid in the composition, the ratio between the at least one carbohydrate to the at least one nucleic acid may be reduced by one or two orders of magnitude.

The complex between the at least one nucleic acid and the at least one cationic polymer is formed into material nanoparticles, wherein each nanoparticle being nanometer in size (nanometer in diameter where the nanoparticles are spherical in shape or have a nanometer axis wherein the nanoparticles are not spherical in shape) and each comprising the at least one nucleic acid, at least one cationic polymer and optionally a small amount of the at least one carbohydrate. The nanoparticles formed in the pre-lyophilized composition and are present also in the lyophilized composition and further in the reconstituted formulation are between about 40 to about 50 nm in size in the pre-lyophilized composition, while in the reconstituted composition the nanoparticle size ranges from about 80 to about 90 nm. Larger nanoparticles are obtained when higher concentrations are utilized, as detailed hereinbelow.

The addition of the nucleic acid/carbohydrate solution into the cationic polymer/carbohydrate solution may be carried out at room temperature, or at any desired temperature, depending, inter alia, on the specific components utilized, the volume of the compositions and other parameters. In some embodiments, the addition is at a constant rate and under constant mixing to form a combined nucleic acid/cationic polymer solution. In some embodiments, the rate is modified for each volume being prepared and the determination of a suitable constant rate is within the routine level of skill in the art.

In some embodiments, the addition, as noted above, is carried out at a rate between 2 and 7 ml/min for small preparations or may be 80 ml/min for a 1 liter preparation, or may vary (increase or decrease) depending on the volume of the composition/formulation or preparation to be prepared. Greater rates may also be employed.

For example, 1 liter of the polyplex containing 100 ml of a nucleic acid, e.g., a plasmid at an initial concentration of 4 mg/ml is used for a total of 0.4 g in a one liter solution. The one liter of solution contains 100 g carbohydrate, e.g., trehalose. Thus, in this example, the ratio of the weight of trehalose in 1 liter solution to the weight of DNA in the one liter solution is 100/0.4 or 250.

In another example, the amount of the nucleic acid is between about 2.5 ml and about 100 ml and the first effective amount of the 10% w/v trehalose solution is between about 10 ml and about 400 ml. Additionally (or alternatively), the amount of the cationic polymer is between about 1.2 ml and about 48 ml and the second effective amount of a 10% w/v trehalose solution is between about 11.3 ml and about 452 ml.

As may be understood various volumes of the compositions or solutions of the invention may be prepared, e.g., between about 25 ml and about 1,000 ml; such as, 25, 50, 75, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, or 1,000 ml. Larger volumes or smaller volumes or intermediate volumes may also be prepared.

Those skilled in the art will recognize that the methods described herein can readily be modified to form volume of the combined nucleic acid/cationic polymer solution that are much higher than 1,000 ml, e.g., 1,000, 1,500, 2,000, 2,500, 3,000, 3,500, 4,000, 4,500, 5,000, 5,500, 6,000, 6,500, 7,000, 7,500, 8,000, 8,500, 9,000, 9,500, 10,000 ml or more.

Unlike many of the compositions of the art, the compositions prepared in accordance with methods of the invention, comprising nucleic acid/cationic polymer solutions form homogenous suspensions. As used herein, a “composition”, “formulation”, “preparation” of the invention may be in a liquid form, e.g., as a solution, suspension or dispersion, or in a solid form, optionally lyophilized. In some embodiments, where the composition is a liquid composition, it is in the form of a water suspension. In some embodiments, the liquid composition may be generally in the form of a suspension with an amount of any one of the composition components being fully or partially soluble. In some embodiments, where the composition is in a solid form, it is a lyophilized composition of matter.

The methods of the invention may optionally comprise a step of lyophilizing a composition of the invention in order to afford a lyophilized composition. The lyophilized composition may be reconstituted immediately prior to use.

Thus, a method according to the invention may be free of a lyophilizing step, in which case, the composition or formulation that is obtained is a pre-lyophilized composition or formulation. If a lyophilization step is necessary or desired, the composition may be treated under such conditions as known in the art for lyophilization of a wet composition.

In some embodiments, the method of the invention comprises a lyophilization cycle that includes freezing the solution at a temperature below 0° C. In some embodiments, the temperature is between −50° C. and 0° C., between −45° C. and 0° C., between −40° C. and 0° C., between −35° C. and 0° C. In some embodiments, lyophilization is achieved at a temperature of about −45±5° C.

In some embodiments, the method of the invention comprises a lyophilization cycle that includes freezing the solution for a period of at least 12 hours, at least 20 hours, at least 24 hours, at least 30 hours, at least 36 hours, at least 48 hours, at least 52 hours, at least 60 hours, at least 66 hours, at least 72 hours.

In some embodiments, the solution is lyophilized at least between 24 and 72 hours.

In some embodiments, the method of the invention comprises a lyophilization cycle that includes freezing the solution at a temperature of about −45±5° C. for at least 12 hours, at least 20 hours, at least 24 hours, at least 30 hours, at least 36 hours, at least 48 hours, at least 52 hours, at least 60 hours, at least 66 hours, at least 72 hours.

The lyophilized composition of matter can be reconstituted using any method(s) known in the art to produce a reconstituted composition of matter. For example, it can be reconstituted by adding an appropriate volume of double distilled water DDW or IV water for injection. Typically, the volume added to the lyophilized composition of matter is the volume that was initially added to the vial prior to lyophilization.

The nanoparticles for the pre-lyophilized composition of matter prepared according to the methods described herein range from about 40 to about 50 nm, while the nanoparticles for the reconstituted composition of matter range from about 80 to about 90 nm.

The complex formed between the nucleic acid and the cationic polymer may be such that the w/w ratio of the carbohydrate to the nucleic acid-cationic polymer in the complex may vary depending, inter alia, on the specific carbohydrate and nucleic acid utilized to form the composition. In some embodiments, the ratio is between 50 and 5,000.

In some embodiments, the ratio is between 50 and 4,000. In some embodiments, the ratio is between 50 and 3.000. In some embodiments, the ratio is between 50 and 2,000. In some embodiments, the ratio is between 50 and 1,000. In some embodiments, the ratio is between 50 and 500.

In some embodiments, the ratio is about 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, or 500.

In some embodiments, the ratio is about 950, 955, 960, 965, 970, 975, 980, 985, 990, 995, 1,000, 1,005, 1,010, 1,015, 1,020, 1,025, 1,030, 1,035, 1,040, 1,045, 1,050, 1,055, 1,060, 1,065, 1,070, 1,075, 1,080, 1,085, 1,090, 1,095, 2,000, 2,005, 2,010, 2,015, 2,020, 2,025, 2,030, 2,035, 2,040, 2,045, 2,050, 2,055, 2,060, 2,065, 2,070, 2,075, 2,080, 2,085, 2,090, 2,095, 3,000, 3,005, 3,010, 3,015, 3,020, 3,025, 3,030, 3,035, 3,040, 3,045, 3,050, 3,055, 3,060, 3,065, 3,070, 3,075, 3,080, 3,085, 3,090, 3,095, 4,000, 4,005, 4,010, 4,015, 4,020, 4,025, 4,030, 4,035, 4,040, 4,045, 4,050, 4,055, 4,060, 4,065, 4,070, 4,075, 4,080, 4,085, 4,090, 4,095, or 5,000.

In some embodiments, the ratio is about 125 or 250 or 500 or 1,000.

In some embodiments, the ratio is below 1,000. In some embodiments, the ratio is 125 or 500.

In another aspect, the invention provides products derived from method of the invention.

In another aspect, the invention provides a lyophilized composition of matter comprising at least one nucleic acid, at least one cationic polymer and at least one carbohydrate, wherein the at least one nucleic acid and the at least one cationic polymer form a complex, such that the w/w ratio of the at least one carbohydrate to the nucleic acid-cationic polymer is between 50 and 5,000.

The products of the invention, those prepared by processes of the invention and those prepared by other processes, and are novel per se, may be pre-lyophilized, lyophilized and reconstituted compositions or solutions comprising at least one nucleic acid, at least one cationic polymer and at least one carbohydrate, wherein the at least one nucleic acid and the at least one cationic polymer form a complex, such that the w/w ratio of the at least one carbohydrate to the nucleic acid-cationic polymer is between 50 and 5,000.

The term “pre-lyophilized composition” and the like refers to an intermediate prepared according to the methods described herein. Specifically, the pre-lyophilized composition of matter is prepared in the “reverse” order where the nucleic acid is added to the polymer. Such a “pre-lyophilized composition of matter” includes the nucleic acid (e.g., the DNA), the polymer (e.g., PET), and the carbohydrate solution (e.g., trehalose).

The term “lyophilized composition” and the like refers to the dry material (i.e., the pre-lyophilized composition of matter following lyophilization).

The term “reconstituted composition of matter” and the like refers to the lyophilized composition of matter and the liquid carrier (e.g., DDW or TV water for injection) used for reconstitution. Typically, the reconstituted composition of matter is reconstituted by the medical practitioner, e.g., physician prior to administration to the patient.

In some embodiments, the composition is a pre-lyophilized composition or solution comprising also liquid medium, e.g., water. In some embodiments, the lyophilized composition is a dry composition being water-free.

The lyophilized compositions of matter described herein are an amorphous powder. The pre-lyophilized and reconstituted (i.e., after reconstitution in water for injection) compositions of matter remain slightly white opalescent in color (i.e., they do not show signs of degradation).

Moreover, the pre-lyophilized composition of matter can be lyophilized for long-term storage periods using any lyophilization methods known in the art or described herein in order to produce lyophilized compositions of matter. Following lyophilization, the lyophilized composition of matter has a shelf life of at least 12 months (e.g., at least 12, 13, 14, 15, 16, 17, 18, 19, 20, 21, 22, 23, 24, or more months).

The lyophilized compositions of matter can be reconstituted prior to use using any methods known in the art or described herein in order to provide a reconstituted composition of matter. Following reconstitution, the reconstituted compositions remain slightly white opalescent in color (i.e., do not show signs of degradation).

The at least one nucleic acid, which may be formulated into a composition or formulation of the invention, is any nucleic acid containing molecule, including DNA or RNA. The term “nucleic acid” also encompasses sequences that include any of the known base analogs of DNA and RNA such as 4-acetylcytosine, 8-hydroxy-N6-methyladenosine, aziridinylcytosine, pseudoisocytosine, 5-(carboxyhydroxyl-methyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyl adenine, 1-methylpseudouracil, 1-methyl guanine, 1-methyl inosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxy-aminomethyl-2-thiouracil, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, uracil-5-oxyacetic acid, queosine, 2-thiocytosine and 2,6-diaminopurine.

In some embodiments, the nucleic acid is a plasmid, namely a polynucleotide containing a regulatory sequence operably linked to a heterologous sequence encoding a cytotoxic gene product, wherein the regulatory sequence is from a genomically imprinted gene that is specifically expressed in cancer cells.

In some embodiments, the term “plasmid” as used herein is meant to refer to any nucleic acid (i.e., DNA, shRNA, siRNA, oligonucleotide, etc.), as defined.

In some embodiments, the at least one nucleic acid is a plasmid recited in PCT/IL1998/000486 (WO 1999/018195), or any US application derived therefrom, herein incorporated by reference.

In some embodiments, the at least one nucleic acid is a plasmid recited in PCT/IL2008/001405 (WO 2009/053982), or any US application derived therefrom, herein incorporated by reference.

In some embodiments, the at least one nucleic acid is a plasmid recited in PCT/IL2006/001110 (WO 2007/034487), or any US application derived therefrom, herein incorporated by reference.

In some embodiments, the at least one nucleic acid is a plasmid recited in PCT/IL2006/000785 (U.S. Pat. No. 8,067,573), or any US application derived therefrom, herein incorporated by reference.

In some embodiments, the at least one nucleic acid is a plasmid recited in PCT/IL2008/000071 (U.S. Pat. No. 7,928,083), or any US application derived therefrom, herein incorporated by reference.

In some embodiments, the regulatory sequence in the plasmid may be an H19 regulatory sequence (e.g., the 1119 promoter, the H19 enhancer, or both the H19 promoter and H19 enhancer). For example, the H19 regulatory sequence may include the H19 promoter and enhancer, and the heterologous sequence encodes a protein selected from the group consisting of β-galactosidase, diphtheria toxin, Pseudomonas toxin, ricin, cholera toxin, retinoblastoma gene, p53, herpes simplex thymidine kinase, varicella zoster thymidine kinase, cytosine deaminase, nitroreductase, cytochrome p-450 2B1, thymidine phosphorylase, purine nucleoside phosphorylase, alkaline phosphatase, carboxypeptidases A and G2, linamarase, β-lactamase, and xanthine oxidase.

In other embodiments, wherein the regulatory sequence is an IGF-2 P4 promoter or an IGF-2 P3 promoter.

Suitable plasmids for use in the methods described herein may include a polynucleotide containing a regulatory sequence operably linked to a heterologous sequence encoding a cytotoxic gene product, wherein the regulatory sequence is from a genomically imprinted gene that is specifically expressed in cancer cells.

The regulatory sequence may be an H19 regulatory sequence (e.g., the H19 promoter, the H19 enhancer, or both the H19 promoter and H19 enhancer), an IGF-2 P4 promoter, or an IGF-2 P3 promoter. For example, the H19 regulatory sequences may be the H19 promoter and enhancer, and the heterologous sequence encodes a protein selected from the group consisting of β-galactosidase, diphtheria toxin, Pseudomonas toxin, ricin, cholera toxin, retinoblastoma gene, p53, herpes simplex thymidine kinase, varicella zoster thymidine kinase, cytosine deaminase, nitroreductase, cytochrome p-450 2B1, thymidine phosphorylase, purine nucleoside phosphorylase, alkaline phosphatase, carboxypeptidases A and G2, linamarase, β-lactamase, and xanthine oxidase. The H19 enhancer may be placed 3′ to the heterologous sequence.

Those skilled in the art will recognize that heterologous sequence may be selected from any one or more of the following: the coding sequence for β-galactosidase; diphtheria toxin; Pseudomonas toxin; ricin; cholera toxin; retinoblastoma gene; p53; herpes simplex thymidine kinase; varicella zoster thymidine kinase; cytosine deaminase; nitroreductase; cytochrome p-450 2B1; thymidine phosphorylase; purine nucleoside phosphorylase; alkaline phosphatase; carboxypeptidases A and G2; linamarase; β-lactamase; xanthine oxidase; and an antisense sequence that specifically hybridizes to a sequence encoding a gene selected from the group consisting of cdk2, cdk8, cdk21, cdc25A, cyclinD1, cyclinE, cyclinA, cdk4, oncogenic forms of p53, c-fos, c-jun, Kr-ras and Her2/neu. The heterologous sequence may also encode a ribozyme that specifically cleaves an RNA encoding a gene selected from the group consisting of cdk2, cdk8, cdk21, cdc25A, cyclinD1, cyclinE, cyclinA, cdk4, oncogenic forms of p53, c-fos, c-jun, Kr-ras and Her2/neu.

The concentration of the nucleic acid within the pre-lyophilized and reconstituted compositions of matter described herein may be between 0.1 mg/mL and 0.8 mg/mL (e.g., 0.1, 0.2, 0.3, 0.4, 0.5, 0.6, 0.7, or 0.8 mg/mL), or lower. This nucleic acid concentration is approximately 8 to 40 times greater than the nucleic acid load seen in prior art compositions (i.e., about 0.01-0.05 mg/mL). In the pre-lyophilized and reconstituted compositions of matter described herein, both the nucleic acid and the PEI are diluted into a trehalose solution.

The at least one carbohydrate utilized in a process and formulations of the invention is any carbohydrate material as known in the art. As used herein, a “carbohydrate” is meant to include any compound with the general formula (CH₂O)ₙ and may interchangeably be used with the term “saccharide”, “polysaccharide”, “oligosaccharide” and “sugar”, as are well known in the art of carbohydrate chemistry.

The carbohydrate may be any one of mono- di-, tri- and oligo-saccharides, as well polysaccharides such as glycogen, cellulose, and starches.

In some embodiments, the at least one carbohydrate is selected from monosaccharides such as glucose, fructose, mannose, xylose, arabinose, galactose, and others; from disaccharides such as trehalose, sucrose, cellobiose, maltose, lactose and others; oligosaccharides such as raffinose, stacchyose, maltodextrins and others; polysaccharides such as cellulose, hemicellulose, starch and others.

In some embodiments, the at least one carbohydrate is selected from trehalose, glucose, sucrose, lactose, mannitol, sorbitol, raffinose, PVP, and dextrose.

In some embodiments, the at least one carbohydrate is not glucose or sucrose.

In some embodiments, the at least one carbohydrate is a monosaccharide or a disaccharide.

In some embodiments, the at least one carbohydrate is trehalose.

The term “cationic polymer” is any polymer, natural, synthetic or semi-synthetic, that comprises cationic groups and/or groups that can be ionized to cationic groups. The cationic polymer may be hydrophilic or amphiphilic. In some embodiments, the cationic polymers are selected from polymers containing primary, secondary, tertiary and/or quaternary amine groups. The amine groups may be part of the main polymer chain or may be pendant on the chain, or may be associated with the chain via one or more side groups connected thereto.

In some embodiments, the at least one cationic polymer is selected from polyethyleneimine, polyallylamine, polyetheramine, polyvinylpyridine, polysaccharides having a positively charged functionalities thereon, polyamino acids, poly-L-histidine, poly-D-lysine, poly-DL-lysine, poly-L-lysine, poly-e-CBZ-O-lysine, poly-e-CBZ-DL-lysine, poly-e-CBZ-L-lysine, poly-OL-ornithine, poly-L-ornithine, poly-DELTA-CBZ-DL-ornithine, poly-L-arginine, poly-DL-alanine-poly-L-lysine, poly(-L-histidine, L-glutamic acid)-poly-DL-alanine-poly-L-lysine, poly(L-phenylalanine, L-glutamic acid)-poly-DL-alanine-poly-L-lysine, poly(L-tyrosine, L-glutamic acid)-poly-DL-alanine-poly-L-lysine, copolymers of L-arginine with tryptophan, tyrosine, or serine, copolymers of D-glutamic acid with D-lysine, copolymers of L-glutamic acid with lysine, ornithine, or mixtures of lysine and ornithine, and poly-(L-glutamic acid).

In some embodiments, the at least one cationic polymer is polyethylenimine (PEI).

Without being bound by theory, it is believed that in the pre-lyophilized, lyophilized, and reconstituted compositions of matter described herein, the three components (i.e., plasmid, the cationic polymer, and carbohydrate) are combined together to provide a novel material form. In contrast, in the prior art compositions of matter, the carbohydrate is added to the previously formed complex and serves only as a both a cryoprotectant and a stabilizing agent, thereby requiring higher ratios as compared to that of the present invention. Thus, the ratio of carbohydrate to nucleic acid in the compositions of matter described herein is approximately about 40 to about 400 times lower than that used in the prior art.

In some embodiments, the ratio of moles of the amine groups of the PEI to the moles of the phosphate groups of the nucleic acid is between 2 and 10 (e.g., 2, 3, 4, 5, 6, 7, 8, 9, 10, or between 6 and 8).

In some embodiments, the compositions of matter do not contain histidine and/or sodium chloride.

In these compositions of matter, the positively charged PEI and the negatively charged nucleic acid form nanoparticles in the presence of the carbohydrate (e.g., trehalose). The nanoparticles for the pre-lyophilization solution range from about 40 to about 50 nm (e.g., about 40, 41, 42, 43, 44, 45, 46, 47, 48, 49, or 50 nm), and the nanoparticles for the reconstituted product range from about 80 to about 90 nm (e.g., about 80, 81, 82, 83, 84, 85, 86, 87, 88, 89, or 90 nm).

Generally speaking, and depending, inter alia, on the positively charged PEI and the negatively charged nucleic acid making up the nanoparticles, the nanoparticle size may very from between 40 nm to about 500 nm. Thus, the nanoparticles may have a size selected from 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495 and 500.

The invention further contemplates formulations as disclosed herein.

Further contemplates are uses of compositions, formulations and preparations according to the invention. In some embodiments, the compositions, formulations and preparations according to the invention may be formulated for use in medicine. Thus, the invention further provides pharmaceutical compositions, formulations and preparations.

The invention further provides use of a composition, formulations or preparation according to the invention in the preparation of a medicament.

In some embodiments, the medicament is for use in a method of treatment of a subject suffering from a disease or disorder treatable by one or more nucleic acids employed in accordance with the invention.

In some embodiments, the disease or disorder treatable by one or more nucleic acids is selected from proliferative diseases and disorders.

In some embodiments, the disease is Rheumatoid arthritis.

Thus, also provided are methods of treating or preventing at least one proliferative disease or disorder and for treating or preventing Rheumatoid arthritis.

The at least one proliferative disease or disorder may be selected amongst cancers. Thus, the invention contemplates further uses in the treatment or prevention of a tumor in a patient. A general method of treatment according to the invention may involve obtaining a lyophilized composition and reconstituting the composition, e.g., using double distilled water (DDW) or TV water for injection, to an effective amount of the composition, and administering the reconstituted composition to the subject.

In some embodiments, where the proliferative disease is cancer, it may be selected from the group consisting of bladder carcinoma, hepatocellular carcinoma, hapatoblastoma, rhabdomysarcoma, ovarian carcinoma, cervical carcinoma, lung carcinoma, breast carcinoma, squamous cell carcinoma in head and neck, esophageal carcinoma, thyroid carcinoma, astrocytoma, ganglioblastoma, and neuroblastoma.

In one embodiment, the bladder carcinoma is non-muscle invasive bladder cancer and the composition is administered intravesically, intravenously, intra-tumorally, or using any other suitable method(s) known in the art.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and are not intended to be limiting.

Other features and advantages of the invention will be apparent from the following detailed description and claims.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-B are graphical representations of assays demonstrating potency of solutions before lyophilization (the pre-lyophilized composition, FIG. 1A) and after reconstitution (the reconstituted, FIG. 1B). Luc 4=no potency (baseline; maximum luminescence; all cells are viable); Comp=potency for the conventional preparation; Rev=potency for the new preparation methodology of the invention; and Usual=fresh composition.

FIG. 2 shows the results of electrophoresis of samples. Lane 1 is the marker; Lane 2 is plasmid BC-819; Lane 3 is BC-819/in vivo-jetPEI® composition in 5% glucose (standard preparation); Lane 4 is a composition in trehalose (reverse protocol); Lane 5 is the lyophilized composition after reconstitution with DDW (the reconstituted composition of matter); and Lane 6 is BC-819/in vivo-jetPEI® composition in 5% trehalose.

FIG. 3 is a graph showing size distribution by volume of particles of PEI/BC-819 compositions prepared using both the standard method and the reverse method described herein.

FIG. 4 is a graph showing zeta potential distribution of particles of PEI/BC-819 compositions prepared using both the standard method and the reverse method described herein.

FIG. 5 is a graph showing the results of the spectrophotometry tests described in Example 3, infra.

FIG. 6 presents the results of electrophoresis of additional samples using the methods described in Example 3, infra. Lane 1 is the ladder; Lane 2 is the BC-819 plasmid; Lane 3 is the composition in glucose solution (standard protocol); Lane 4 is a composition in trehalose (reverse protocol); Lane 5 is a lyophilized polyplex after rehydration (the reconstituted composition); and Lane 6 is a composition in trehalose solution.

FIGS. 7A-B are TEM pictures showing the appearance of a glucose composition (FIG. 7A) and a trehalose composition (FIG. 7B).

FIG. 8 is a TEM picture showing a sample after composition precipitation.

FIGS. 9A-B are pictures showing results of transmission electron microscopy (TEM).

FIG. 10 is a graph showing tumor progression of HCT-116 cells in nude mice receiving three injections of the prior art composition of matter or reconstituted lyophilized composition compared to a 5% glucose control.

### DETAILED DESCRIPTION OF EMBODIMENTS

As used herein, the terms “glucose polyplex”, “glucose composition of matter”, and the like refer to prior art compositions of matter that are prepared using glucose as the polysaccharide rather than trehalose. Likewise, the terms “standard polyplex preparation”, “standard composition of matter”, and the like refer to prior art compositions of matter that are prepared by addition the polymer to the plasmid (i.e., the “non-reverse” order).

As used herein, the terms “aggregation”, “aggregate”, and the like refer to particles having a size larger than the highest particle size that is deemed to be acceptable. To determine the highest acceptable particle size, different measures of light scattering are provided and the standard preparation and the new preparation methods are compared to see if there are any differences.

Regulatory sequences that can be used to direct the tumor cell specific expression of a heterologous coding sequence are known in the art. For example, H19 regulatory sequences, including the upstream H19 promoter region and/or the downstream H19 enhancer region are described in U.S. Pat. No. 6,087,164, which is herein incorporated by reference in its entirety. The downstream enhancer region of the human H19 gene can optionally be added to an H19 promoter/heterologous gene construct in order to provide enhanced levels of tumor cell-specific expression.

U.S. Pat. No. 6,087,164 also describes the use of the IGF-2 P3 and P4 promoters in combination with the H19 enhancer or active fragments thereof.

The skilled artisan will be able to use regulatory sequences from genomically imprinted and non-imprinted genes that are expressed in cancer cells in order to direct tumor specific expression of heterologous coding sequences in appropriate host cells, for example, H19-expressing carcinoma cells (e.g. bladder carcinoma cells, to name an example). Any altered regulatory sequences which retain their ability to direct tumor specific expression be incorporated into recombinant expression vectors for further use.

A wide variety of heterologous genes can be expressed under the control of these regulatory sequences such as genes encoding toxic gene products, potentially toxic gene products, and antiproliferation or cytostatic gene products. Marker genes can also be expressed including enzymes, (e.g. CAT, beta-galactosidase, luciferase), fluorescent proteins such as green fluorescent protein, or antigenic markers.

Cytotoxic gene products are broadly defined to include both toxins and apoptosis-inducing agents. Additionally, cytotoxic gene products include drug metabolizing enzymes which convert a pro-drug into a cytotoxic product. Examples of cytotoxic gene products that may be used in methods of the invention comprise diphtheria toxin, Pseudomonas toxin, ricin, cholera toxin, PE40 and tumor suppressor genes such as the retinoblastoma gene and p53. Additionally, sequences encoding apoptotic peptides that induce cell apoptosis may be used. Such apoptotic peptides include the Alzheimer's A beta peptide (see LaFerla et al., Nat. Genet. 9:21-30 (1995)), the atrial natriuretic peptide (see Wu et al., J. Biol. Chem. 272:14860-14866 (1997)), the calcitonin gene-related peptide (see Sakuta et al., J. Neuroimmunol. 67:103-109 (1996)), as well as other apoptotic peptides known or to be discovered.

Drug metabolizing enzymes which convert a pro-drug into a cytotoxic product include thymidine kinase (from herpes simplex or varicella zoster viruses), cytosine deaminase, nitroreductase, cytochrome p-450 2B1, thymidine phosphorylase, purine nucleoside phosphorylase, alkaline phosphatase, carboxypeptidases A and G2, linamarase, .beta.-lactamase and xanthine oxidase (see Rigg and Sikora, Mol. Med. Today, pp. 359-366 (August 1997) for background).

Additionally, antisense, antigene, or aptameric oligonucleotides may be delivered to cancer cells using expression constructs. Ribozymes or single-stranded RNA can also be expressed in the cancer cell to inhibit the expression of a particular gene of interest. The target genes for these antisense or ribozyme molecules should be those encoding gene products that are essential for cell maintenance or for the maintenance of the cancerous cell phenotype. Such target genes include but are not limited to cdk2, cdk8, cdk21, cdc25A, cyclinD1, cyclinE, cyclinA and cdk4.

For example, vectors which express, under the control of regulatory sequences from imprinted genes or IGF-1 promoter that are expressed in cancer cells, antisense RNAs or ribozymes specific for the transcripts of oncogenic forms of p53, c-fos, c-jun, Kr-ras and/or Her2/neu are introduced into cells in order to down-regulate expression of the endogenous genes. Tumor cells which express H19, and can activate the H19 regulatory sequences, (or which specifically activate IGF-1, the IGF-2 P3 or P4 promoter) can be specifically targeted for expression of the antisense RNA or ribozyme RNA.

Antisense approaches involve the design of oligonucleotides (in this case, mRNA) that are complementary to the target mRNA. The antisense oligonucleotides will bind to the complementary target mRNA transcripts and prevent translation. Absolute complementarity is not required. A sequence “complementary” to a portion of an RNA, as referred to herein, means a sequence having sufficient complementarity to be able to hybridize with the RNA, forming a stable duplex. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the duplex.

Oligonucleotides that are complementary to the 5′ end of the target message, e.g., the 5′ untranslated sequence up to and including the AUG initiation codon, should work most efficiently at inhibiting translation. However, sequences complementary to the 3′ untranslated sequences of mRNAs have recently shown to be effective at inhibiting translation of mRNAs as well. See generally, Wagner, R., Nature 372:333-335 (1994). Thus, oligonucleotides complementary to either the 5′- or 3′-non-translated, non-coding regions of the target gene transcripts could be used in an antisense approach to inhibit translation of endogenous genes. Oligonucleotides complementary to the 5′ untranslated region of the mRNA should include the complement of the AUG start codon. Antisense oligonucleotides complementary to mRNA coding regions are less efficient inhibitors of translation but could be used in accordance with the invention. Whether designed to hybridize to the 5′, 3′ or coding region of the target mRNA, antisense nucleic acids should be at least six nucleotides in length, and are preferably oligonucleotides ranging from 6 to about 50 nucleotides in length. For example, the oligonucleotide is at least 10 nucleotides, at least 17 nucleotides, at least 25 nucleotides or at least 50 nucleotides.

Ribozyme molecules designed to catalytically cleave an essential target gene can also be used to prevent translation of target mRNA. (See, e.g., PCT International Publication WO90/11364, published Oct. 4, 1990; Sarver et al., Science 247:1222-1225 (1990)). When the ribozyme is specific for a gene transcript encoding a protein essential for cancer cell growth, such ribozymes can cause reversal of a cancerous cell phenotype. While ribozymes that cleave mRNA at site specific recognition sequences can be used to destroy target mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. Construction and production of hammerhead ribozymes is well known in the art and is described more fully in Haseloff and Gerlach, Nature, 334:585-591 (1988). Preferably the ribozyme is engineered so that the cleavage recognition site is located near the 5′ end of the target mRNA; i.e., to increase efficiency and minimize the intracellular accumulation of non-functional mRNA transcripts.

Ribozymes also include RNA endoribonucleases (hereinafter “Cech-type rihozymes”) such as the one which occurs naturally in Tetrahymena thermophila (known as the IVS, or L-19 IVS RNA) and which has been extensively described by Thomas Cech and collaborators (Zaug et al., Science, 224:574-578 (1984); Zaug and Cech, Science, 231:470-475 (1986); Zaug et al., Nature, 324:429-433 (1986); published International Patent Application No. WO 88/04300 by University Patents Inc.; Been and Cech, Cell, 47:207-216 (1986)). The Cech-type ribozymes have an eight base pair active site which hybridizes to a target RNA sequence where after cleavage of the target RNA takes place.

Any plasmids known in the art can be used in the methods and compositions of the invention. By way of non-limiting example, the plasmids BC-819 and BC-821 (BioCancell, Israel) can be used. These plasmids are described in more detail in U.S. Pat. No. 6,087,164 and U.S. Published Patent Application No. 20100256225, which are herein incorporated by reference in its entirety.

Cells that reactivate imprinted gene expression will also be capable of specifically activating expression constructs containing such imprinted gene regulatory regions operatively linked to a heterologous gene. Such cells, particularly tumor cells, are appropriate targets for the gene therapy methods of the invention. H19, and IGF-2 P3 and P4 specific expression in both tumors and cell lines may be determined using the techniques of RNA analysis, in situ hybridization and reporter gene constructs. In addition, tumor cells with activated IGF-1 gene expression may be similarly determined and targeted in gene therapy using the IGF-1 promoter to direct expression of a heterologous gene.

Exemplary tumor types with activated H19 expression are as follows:

A. Pediatric solid tumors
1. Wilm's tumor
2. Hepatoblastoma
3. Embryonal rhabdomyosarcoma

B. Germ cell tumors and trophoblastic tumors
1. Testicular germ cell tumors
2. Immature teratoma of ovary
3. Sacrococcygeal tumor
4. Choriocarcinoma
5. Placental site trophoblastic tumors

C. Epithelial adult tumors
1. Bladder carcinoma
2. Hepatocellular carcinoma
3. Ovarian carcinoma
4. Cervical carcinoma
5. Lung carcinoma
6. Breast carcinoma
7. Squamous cell carcinoma in head and neck
8. Esophageal carcinoma
9. Thyroid carcinoma

D. Neurogenic tumors
1. Astrocytoma
2. Ganglioblastoma
3. Neuroblastoma

Any of these cancers are treatable by the methods of the invention. In fact, any tumors which activate H19 expression may be treated by the methods of the invention. Additionally, tumors that activate the IGF-1, and the IGF-2 P3 and P4 promoters are also treatable by the methods of the invention. For example, IGF-2 P3 and P4 promoters are activated in childhood tumors, such as Wilm's tumors, rhabdomyosarcomas, neuroblastomas and hepatoblastomas.

Therapy

The invention also encompasses the use of polynucleotides containing a regulatory region operatively linked to a heterologous gene for use in therapy to treat cancer and hyperproliferative diseases. For therapy purposes, expression constructs of the instant invention may be administered in any biologically effective carrier, e.g., any formulation or composition capable of effectively delivering the nucleotide construct to cells in vivo.

In addition to viral transfer methods, non-viral methods can also be employed to cause directed expression of a desired heterologous gene in the tissue of an animal. Most non-viral methods of gene transfer rely on normal mechanisms used by mammalian cells for the uptake and intracellular transport of macromolecules. In some embodiments, non-viral gene delivery systems of the present invention rely on endocytic pathways for the uptake of the subject expression constructs by the targeted cell. Exemplary gene delivery systems of this type include, for example, polyplexes.

Polyplex Formation and Lyophilization

As described herein, cationic polymers can be used as a transfection reagent in combination with a nucleic acid, e.g., a plasmids in order to mediate efficient nucleic acid (e.g., DNA, shRNA, siRNA, oligonucleotide, etc.) delivery into cells and tissues. Compositions of matter are formed when nucleic acids complex with the cationic polymers.

Those skilled in the art will recognize that other additives commonly used in the art, such as, for example, lipids, liposomes, cholesterol, polyethyleneglycol (PEG), micelles, hyaluronic acid, proteins, emulsifying agents, surfactants, viral vectors, and/or targeting moieties may also be utilized in compositions of the invention.

However, in some embodiments, the polyplex formulations (e.g., the pre-lyophilized compositions of matter) contain only the nucleic acid, the cationic polymer, the sugar and an optional buffer. In contrast, other formulations used in the art contain other components such as, for example, histidine.

One exemplary cationic polymer for use in polyplex formation is polyethylenimine (PEI). (See U.S. Pat. No. 6,013,240, incorporated by reference). For example, a linear polyethylenimine (in vivo-jetPEI®, Polyplus-transfection S.A., France) can be used. in vivo-jetPEI® is a 150 mM solution, expressed as nitrogen residues. Alternatively, linear PEI can be prepared by hydrolyzing a commercially available branched PEI or by any other methods known in the art.

Those skilled in the art will recognize that the ionic balance within the cationic polymer/nucleic acid composition of matter is crucial and that, for effective cell entry, the compositions of matter should be cationic. The N/P ratio is defined as the number of nitrogen residues in the cationic polymer per nucleic acid phosphate. Preferably, for in vivo nucleic acid delivery, the N/P ratio is between 2-10 (e.g., between 6-8). Determination of the N/P ratio is within the routine level of skill in the art.

The nucleic acid-PEI composition of matter (e.g., the pre-lyophilized composition of matter) contains nanoparticles formed during the mixing of the PEI, the plasmid DNA, and the carbohydrate. When mixed, the positive charge of the cationic polymer and the negative charge of the plasmid form nanoparticles.

Any suitable delivery route can be used, for example: intravenous (IV), intraperitoneal (IP), intratumoral, subcutaneous, topical, intrathecal, intradermal, intravitreal, intradermal, intracortical, intratesticular, intra-arterial, intravesical (e.g., into the bladder), intraporteal, intracerebral, retro-orbital injection, intranasally, and the like. In some embodiments, the gene delivery vehicle can be introduced by catheter. (See U.S. Pat. No. 5,328,470). Delivery of the nucleic acid in to the cell or tissue can be done in vitro, in vivo, ex vivo, or in situ.

As outlined in Example 1, infra, a standard polyplex formation is accomplished by introduction of the transfection reagent (e.g., in vivo-jetPEI®) diluted in a 5% dextrose solution into the DNA plasmid (e.g., BC-819 plasmid) diluted in a 5% dextrose solution followed by aggressive mixing. This standard process for preparing the plasmid/cationic polymer composition of matter is very sensitive, and, if it is not performed in the correct order, it is liable to result in precipitation. Moreover, the resulting polyplex composition of matter may be unstable and a yellowish color is observed after 3 hours at room temperature. Additionally, when preparing with the standard protocol, previous attempts to reverse the process of adding the plasmid to the transfection reagent did not result in nanoparticle formation. Moreover, these solutions are limited to micro-volumes and require a high ratio of cryoprotectant to nanoparticles to achieve lyophilization.

Thus, methods of polyplex formation are needed that can be scaled up for the high scale preparation and long term storage of well-defined and stable solutions or freeze dried compositions of matter that will ensure a predictable quality and increased stability upon rehydration.

Some prior studies have shown that DNA plasmids/LPEI compositions of matter can be lyophilized in presence of a lyoprotectant in high concentration (i.e., a high ratio of carbohydrate/DNA plasmid w/w) and maintain their initial quality. (See Brus et al., Journal of Controlled Release 95:119-131 (2004)). With PEI, it has been reported that a ratio of sugar/DNA of 7500 protected the complexes. Moreover, cationic lipid-DNA (not LPEI) required a ratio of 250. However, the lyophilization process used in Brus et al. is not reproducible in large scales. Moreover, the high concentration of lyoprotectant used in these studies, might not be tolerated in vivo and, thus, could result in final product that might be irrelevant for use in clinical studies.

In fact, the inventors of the present invention have surprisingly discovered that is possible to use much lower ratios of carbohydrates (sugars) to nucleic acids in the lyophilization process, thereby resulting in compositions of matter with a reduced carbohydrate to nucleic acid ratio. Without being bound by theory, the inventors believe that the three components of the compositions of matter described herein (DNA, Jet-PEI, and carbohydrate) combined to form a new chemical entity that can easily be lyophilized. Therefore, much lower concentrations of carbohydrates are needed.

These lower concentrations are better tolerated for in vivo applications. For example, the normal physiologic range for osmolality of human blood is approximately 280 to 310 mOsmol/L, while compositions of matter prepared in glucose are 250 and compositions of matter in trehalose for use in the bladder are 280. Thus, the solution that is being prepared for bladder is slightly hypotonic, but it is being administered intravesically. For IV preparations, the concentration will be increased to a DNA/trehalose ratio of up to 300.

Those studies have also raised the challenging aspect of preparing high volumes polyplex solutions and reported the development of a micro-mixer system method (see Kasper et al., European Journal of Pharmaceutics and Biopharmaceutics 77:182-185(2011)) to achieve this goal. However, this micro-mixer system still needs up-scaling development to be brought to industrial scale.

Example 3, infra, describes the preparation of pre-lyophilized compositions of matter that are constituted of the non-viral vector BC-819, expressing the diphtheria toxin A chain (DTA) under the control of the H19 gene regulatory sequences, and the in vivo-jetPEI®, in presence of a 5% trehalose solution.

The use of a new DNA-based therapy for cancer treatment in which BC-819 (also known as H19-DTA) and BC-821 plasmids (also known as H19-DTA-P4-IGF2) drive the expression of DTA under the control of the H19 (for BC-819) or H19 and IGF2 P4 (for BC-821) regulatory elements to selectively target cancer cells has previously been reported. (See Ohan a et al., International Journal of Cancer 98(5):645-650 (2002); Ohana et al., The Journal of Gene Medicine 7(3):366-374 (2005); Abraham et al., The Journal of Urology 180(6):2379-2383 (2008)). These therapies demonstrated good results in treatment of colon to liver metastases, bladder, pancreatic and ovarian cancers. (See Ohana et al., The Journal of Gene Medicine 7(3):366-374 (2005); Mizrahi et al., Journal of Translational Medicine 7:69 (2009); Ohana et al., Gene Therapy and Molecular Biology 8:181-192 (2004); Scaiewicz et la., Journal of Oncology 178174 (2010); Sorin et al., International Journal of Oncology 39(6):1407-12 (2011); Abraham et al., The Journal of Urology 180(6):2379-2383 (2008)).

In these bladder cancer clinical trials, BC-819 is administered as a complex with in vivo-jetPEI® (N/P=6) at a final concentration of 0.4 mg/ml in a final volume of 50 ml 5% glucose.

However, the preparation of a good quality composition of matter remains a challenge, due to the strict recommendations for mixing the components and the relative instability of the composition of matter formed at this concentration. (See Kasper et al., European Journal of Pharmaceutics and Biopharmaceutics 77:182-185(2011)).

As described in Example 1, infra, at the patient's bedside, both plasmid and PEI solutions are diluted in 5% w/v glucose solution separately and the PEI solution is then added very fast to the plasmid solution. Any deviation from the protocol may lead to a decrease in the composition of matter quality or even worse: precipitation. Thus, the composition of matter preparation requires highly qualified staff involvement at each dose administration. In addition the resulting polyplex solution has a short shelf life.

Accordingly, an optimal way to ensure the administration of reproducible good quality polyplexes is to provide a ready-for-use product (e.g., the lyophilized composition of matter) with a shelf life of at least 24 months to the pharmacy. This product will need a simple reconstitution with water prior to use.

The methods described herein allow the composition of matter solution to be prepared in high volume at the desired concentration, in, e.g., trehalose solution that can subsequently be lyophilized in therapeutic doses.

The final trehalose concentration used during the preparation provides an isotonic environment that allows for the formation of a stable composition of matter and is tolerated as well as fresh-made solution when administered in vivo.

The formation of a stable pre-lyophilized composition of matter solution is critical to ensure a successful freeze drying process.

Use of this method also allows the technical limit of the high volume polyplex preparations to be overcome by reversing the order of mixing both components. Here, the preparation of an isotonic and stable pre-lyophilized composition of matter solution in relatively high concentration that can go through freeze drying, without altering the product was achieved.

This method is a breakthrough in the generation of Plasmid-LPEI (linear PEI) composition of matter that can be up scaled to an industrial production and will provide a stable product that can be easily stored and uniformly prepared prior administration to patients.

To overcome the limitations of the standard polyplex formation process, provided herein is a pre-lyophilized composition of matter solution formation process where the nucleic acid (e.g., BC-819 plasmid), the cationic polymer (e.g., in vivo-jetPEI®), and the carbohydrate (e.g., trehalose) are mixed in the reverse order (as compared to the standard protocol). This process is referred to interchangeably herein as the “reverse process”, “reverse method”, and/or “reverse polyplex formation”. In this way, the plasmid is added to the transfection reagent in a slow and controlled process accompanied by a continuous mixing of the formed composition of matter solution. Importantly, the pre-lyophilized composition of matter solution is composed of the nucleic acid, LPEI, and the carbohydrate to form a new chemical entity (NCE) that is very stable to ensure a good lyophilized product.

The resulting lyophilized composition of matter contains only three components: the PET, the DNA plasmid, and trehalose. Moreover, the composition of matter is no longer defined as the complexed PEI/plasmid. Rather, it is an amorphous powder that has different chemical structure.

The composition of matter prepared according to the reverse method described herein has a carbohydrate to nucleic acid-polymer composition of matter ratio, which is much lower than the ratio observed when using other polyplex formation methods known in the art.

Lyophilization is the dehydration process of a solubilized compound without heating mediated vaporization. In the lyophilization process a solution is frozen and subjected to low pressure environment, under which water sublimation process is facilitated, with zero to minimal damage to the solubilized compound.

Once the cationic polymer is complexed with the nucleic acid using the reverse protocol, it can be lyophilized in accordance with any lyophilization methods described herein or known in the art. Prior to the instant invention, when adding trehalose, mannitol, or sucrose at the concentration of 10 to 50 μg/mL to the polyplexes, a decrease in efficiency was observed after lyophilization. (See Brus et al., Journal of Controlled Release 95 (2004) 119-131).

The lyophilized compositions of matter can be stored in the lyophilized form until use and reconstituted (e.g., with DDW) prior to injection into patients. The lyophilized composition of matter will have a shelf life greater than 3 months (i.e., greater than 3, 4, 5, 6, 7, 8, 9, 10, 11, 12, 13, 14, 15, 16, 17, 18, 19, 20, 21, 22, 23, 24, or more months).

After reconstitution, the appearance of the composition of matter is similar to that of a fresh composition of matter solution prepared according to the reverse preparation method, and no precipitation is observed. Likewise, the reconstituted samples show no differences from either the standard or the new preparation methods. Importantly, the nucleic acid concentration in the polyplex solution is not affected by the lyophilization process.

The fresh complex prepared by the method of the prior art resulted in particles in the range of 50-500 nm. Preparation of the prelyophilization solution in the reverse order resulted in particles in the range of 40-50 nm. The reconstituted sample after lyophilization (using the reverse order) resulted in particles in the range of 80-90 nm. Thus, following reconstitution, the size of the nanoparticles was comparable to those of the fresh composition of matter. In contrast, as describe in Kasper et al., when prepared using a micro-mixer for low DNA concentration complex, the resulting particles were in the range of 65-170 nm.

After 3 h at room temperature, the fresh composition of matter prepared by the method of the prior art (prepared with glucose) exhibited signs of degradation (i.e., slightly yellowish color). In contrast, both the reconstituted sample and the solution prepared according to the reverse preparation method (the prelyophilized case in trehalose) showed no visible signs of degradation and were slightly white opalescent in color. After 24 h at room temperature, the fresh composition of matter prepared by the method of the prior art (prepared with glucose) was yellow while the reconstituted sample and the solution prepared according to the reverse preparation method (prelyophilization in trehalose) showed no visible signs of degradation and remained slightly white opalescent in color

Accordingly, the use of the reverse preparation method in combination with lyophilization should solve many of the problems associated with the standard process.

For example, at the patient's bedside, the medical practitioner will only have to dissolve the lyophilized powder (the lyophilized composition of matter) in IV water for injection in order to reconstitute the lyophilized composition of matter prior to use, thereby simplifying preparation and administration. Moreover, the shipment of the lyophilized powder is easier due to its expected improved stability. In addition, the properties of the lyophilized product are comparable to the original, hydrous (i.e., non-lyophilized) plasmid.

The reverse polyplex formulation method (pre-lyophilized composition of matter in trehalose) described herein is also different from the methods described in the 2012 doctoral thesis of Julia Christina Kasper entitled “Lyophilization of Nucleic Acid Nanoparticles—Formulation Development, Stabilization Mechanisms, and Process Monitoring.” Kasper recognized the need for a well-defined method for preparing stable polyplexes solutions and understood that the aggregation of the nanoparticles commonly observed when trying to lyophilize polyplexes appears during the freezing step of the lyophilization.

To overcome these issues, Kasper developed a different method to prepare polyplexes. A summary of this method compared to the reverse protocol (prelyophilization in trehalose) described herein is provided in Table 1.

TABLE 1

| | Kasper | Reverse protocol |
|---|---|---|
| Plasmid | pCMVluc (commercial) | BC-819 (BioCancell, Israel) |
| | https:/www.addgene.org/45968/ | |
| Buffer for diluting the Plasmid | 10 mM Histidine buffer pH 6.0 | Tris EDTA pH 8.0 |
| Buffer for diluting the PEI | 10 mM Histidine buffer pH 6.0 | Water for Injection (WFI) |
| PEI | In house hydrolizalion of | In vivo-jetPEI ® |
| | branched PEI | |
| Ratio N/P | 6 | 6 |
| Preparation volume | 0.5 ml | 1 l |
| Preparation method | Micro mixer (method still needs to be | Adding plasmid diluted |
| | developed for larger volumes) | into trehalose solution to the PEI |
| | | diluted into a trehalose solution |
| | | at a rate of 3-5 ml/min while |
| | | stirring at 500 rpm |

Table 2 below compares the technical aspects of the Kasper polyplex preparation and lyophilization protocol and the reverse polyplex preparation and lyophilization methods described herein.

TABLE 2

| | | Reverse Protocol + |
|---|---|---|
| | Kasper | Lyophilization |
| Plasmid | pCMVluc (commercial) | BC-819 (BioCancell, Israel) |
| | https:/www.addgene.org/45968/ | |
| Buffer for diluting the plasmid | 10 mM Histidine buffer pH 6.0 | Tris EDTA pH 8.0 |
| Buffer for diluting the PEI | HBG buffer pH 7.4 (5% glucose | WFI |
| | 20 mM Hepes) or 10 mM | |
| | Histidine buffer pH 6.0 | |
| PEI | In house hydrolization of | In vivo-jetPEI ® |
| | branched PEI | |
| Trehalose addition | Mixed in 10 mM Histidine | Mixed with both |
| | buffer pH 6.0 and added to the | plasmid and PEI at 5% |
| | polyplex 1:1 | |
| Ratio N/P | 6 | 6 |
| Preparation method | Micro mixer (method still needs to be | Adding plasmid diluted |
| | developed for larger volumes) | in trehalose solution |
| | | to the PEI diluted into a |
| | | trehalose solution at a rate of 3-5 |
| | | ml/min while stirring at 500 rpm |
| Final plasmid concentration | 0.1 mg/ml | 0.4 mg/ml |
| Final solution volume | 0.5 ml up to 5 ml | 1 l |
| Solution DSC testing | Yes | Yes |
| Lyophilizer | freeze-drier Lyostar II, SP | freeze dryer LyoBeta 25 |
| | Scientific, Stone Ridge USA | Telstar spain |
| stability | 2-8 C., 20 C., 40 C. for 6 weeks | 2 weeks at 2-8 C. so far |

Kasper also describes the importance of the preparation of a stable composition of matter and the influence of the freezing step in the lyophilization process. Table 3 below summarizes the technical aspects of the Kasper polyplex preparation and lyophilization at Kasper laboratory and the reverse polyplex preparation and lyophilization methods described herein.

TABLE 3

| | Kasper | BioCancell |
|---|---|---|
| Plasmid | pCMVluc (commercial) | BC-819 (BioCancell, Israel) |
| | https:/www.addgene.org/45968/ | |
| Buffer for diluting the plasmid | 10 mM Histidine buffer pH 6.0 | Tris EDTA pH 8.0 |
| Buffer for diluting the PEI | 10 mM Histidine buffer pH 6.0 | WFI |
| PEI | In house hydrolization of | Polyplus (commercial) |
| | branched PEI | |
| Carbohydrate addition | Sucrose Mixed in 10 mM | Trehalose in water for |
| | Histidine buffer pH 6.0 and added to | IV injection Mixed with both |
| | the polyplex 1:1 | plasmid and PEI at |
| | | 5% |
| Carbohydrate/DNA Ratio | 1200 to 14000 | 125 |
| Ratio N/P | 6 | 6 |
| Preparation method | Micro mixer (method still needs to be | Adding plasmid diluted |
| | developed for larger volumes) | into trehalose solution |
| | | to the PEI diluted into a |
| | | trehalose solution at a rate of 3-5 |
| | | ml/min while stirring at 500 rpm |
| Final plasmid concentration | 0.01 mg/ml and 0.05 mg/ml | 0.4 mg/ml |
| Final solution volume | 0.5 ml | 1.1 |
| Freezing method prior drying | Shelf ramp vs “standard” | Shelf ramp |
| | depressurization . . . | |
| Freezing rate at the shelf- | −1 C. or −5 C./min to −45 C. | −1 C./min to −45 C. |
| ramp freezing method | | |
| Particle size | DLS (65 to 170 nm) | DLS (80-90 nm) |
| stability | 2-8 C., 20 C., 40 C. for 6 weeks | 2 weeks at 2-8 C. so far |

As noted in Kasper, the size of the polyplexes is affected by freeze drying: the particle size is better preserved as the ratio of sucrose to plasmid DNA is increased.

Specifically, Kasper teaches that the 0.05 mg/ml DNA solution could be stabilized with sucrose/DNA ratio of at least 2800. According to Kasper, the ratio of stabilizer/DNA is critical to achieve the complete stabilization. The critical ratio depends on the freezing method.

In contrast, no particle aggregation is observed with the reverse polyplex, lyophilization, and reconstitution methods described herein. In addition, the ratio of trehalose to nucleic acid used in these methods is 125 and 250, which is much lower what is described in Kasper.

Additionally, the prelyophilization solution prepared by the reverse method described herein results in compositions of matter (e.g., pre-lyophilized compositions of matter or polyplexes) with a size range of about 40-50 nm. Following reconstitution, the reconstituted composition of matter had a particle size of about 80-90 nm. In contrast, the micro-mixer described in Kasper resulted in polyplexes with a range size of 65-170 nm prior lyophilization. Moreover, Kasper reported a marginal increase in the z-diameter of the polyplexes after lyophilization. Kasper also teaches that the choice of the excipient is of minor importance as long as the viscosity is high enough to avoid particle movement during the freezing phase. The shelf ramp method is less stressful than any other checked method for freezing the compositions of matter.

Accordingly, Kasper concludes that neither plasmid DNA nor siRNA has been successfully lyophilized without limitations. For example, in a first freeze-thaw study, high concentrations of the commonly used disaccharides, sucrose or trehalose, were required to maintain particle size, and these greatly exceeding isotonicity levels, thereby indicating the prerequisite of a critical ratio of stabilizer to polyplex (˜4000). In fact, in Kasper, higher molecular weight excipients, such as lactosucrose, hydroxylpropyl betadex (HP-b-CD), or povidone (PVP), were beneficial for sufficient particle stabilization at low osmotic pressure during freezing and drying. Using isotonic formulations with 14% lactosucrose, 10% HP-b-CD/6.5% sucrose, or 10% PVP/6.3% sucrose, polyplex size was far better preserved (<170 nm) upon lyophilization and storage over 6 weeks up to 40° C. compared to previous studies.

Thus, by using lactosucrose or CD/sucrose formulations, Kasper demonstrated that pDNA/LPEI polyplexes could be lyophilized with only a marginal increase in size and preserved biological activity.

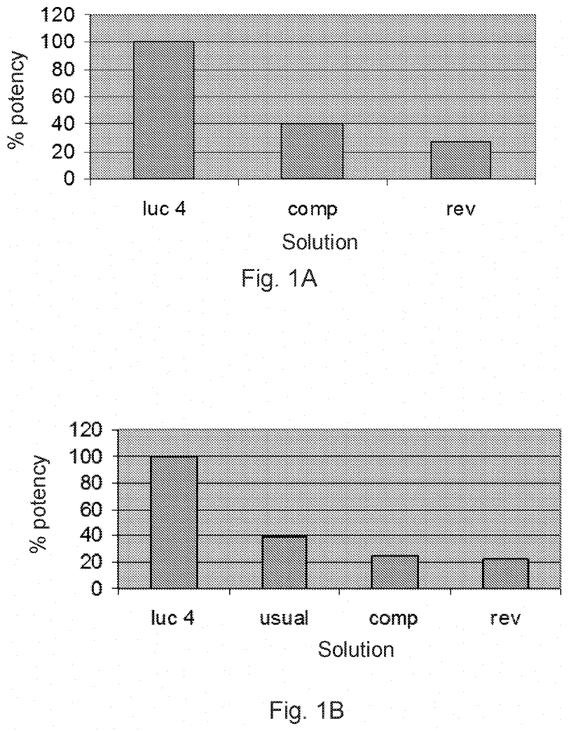

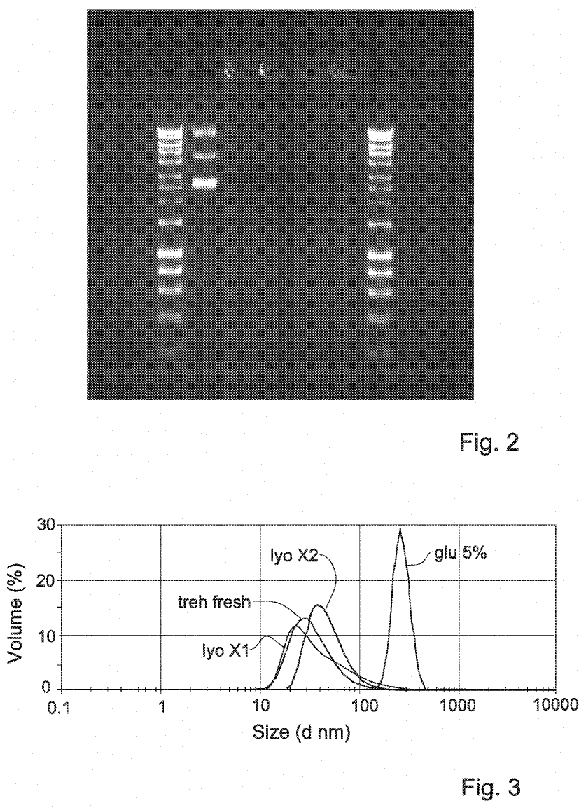

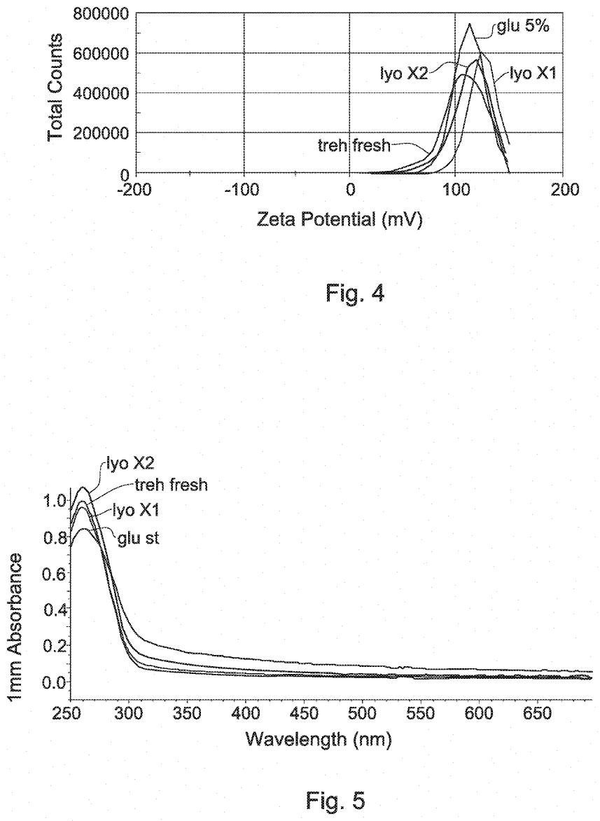

Moreover, Kasper also describes a micro-mixer apparatus that they developed in order to prepare compositions of matter. (See Kasper 2012, FIG. 4-1). This apparatus was used to prepare Plasmid-LPEI compositions of matter in volumes up to 5 ml by mixing the LPEI and the plasmid solutions at the junction of a T-connector. The mixing speed was controlled by using syringes to insert the solutions in the equipment.

The compositions of matter that could be successfully prepared in this manner were up to 50 μg/ml with LPEI at the ratio N/P=6 (molar ratio of LPEI nitrogen (N) to DNA phosphate (P); indicated polyplex concentration, refer to the plasmid DNA concentration of the sample). However, higher plasmid concentration solutions (especially the 400 μg/ml solution) resulted in unstable compositions of matter with high size nanoparticles.

Moreover, in the Kasper method, both plasmid and LPEI were mixed into 10 mM Histidine buffer pH 6, and the composition of matter solution needed additional high concentration excipient in order to stabilize it during the lyophilization process. Specifically, the Kasper carbohydrate/DNA ratio was 1,200 to 14,000.

In contrast, the reverse method described herein is performed in a standard mixing vessel with pinch paddle turbine. This reverse method provides a way to ensure the preparation of large scale polyplex solution (i.e., the pre-lyophilized composition of matter) with a reproducible good quality.

Specifically, as noted above, the mixing order is critical to ensure a high quality pre-lyophilized composition of matter. Here, the LPEI solution and the plasmid are mixed each with a 5% trehalose solution. The PEI solution is then placed in the mixing vessel, and the plasmid solution is added to the LPEI while mixing.

Surprisingly, the insertion of the negatively charged plasmid solution into the highly positively charged LPEI solution in the presence of trehalose results in a homogenous pre-lyophilized composition of matter solution with small nanoparticle size (around 50 nm) and with high stability.

In this way, 1000 ml of a solution at the concentration of 400 μg/ml DNA complexed with in vivo-jetPEI® at the ratio N/P=6 (molar ratio of LPEI nitrogen (N) to DNA phosphate (P); indicated polyplex concentration, refer to the plasmid DNA concentration of the sample) was obtained. The ratio of carbohydrate (trehalose)/DNA is 125.

The resulting solution can then be filled in 100 ml vials and lyophilized to produce lyophilized compositions of matter. In one non-limiting example, the vials went through a freeze drying cycle in a laboratory freeze dryer LyoBeta 25, including a freezing step at −45° C. during at least 4 hours, a primary drying of at least 41 hours, preferably at least 80 hours, more preferably at least 140 hours (e.g., at least 41, 50, 60, 70, 80, 90, 100, 110, 120, 130, or 140 hours) at 25° C., and a secondary drying of at least 8 hours both drying at pressure of 0.150 mb.

Those skilled in the art will recognize that the lyophilized compositions of matter can be reconstituted using any suitable method(s) to form the reconstituted compositions of matter.

Thus far, the Kasper apparatus has been unable to produce composition of matter solutions in big volumes, at a relatively high concentration with a good quality (low size particle, stable composition of matter, etc.). In contrast, the reverse polyplex formation method described herein is simple and results in high quality, stable pre-lyophilized composition of matter solution at relatively high concentrations without the need of excipient to protect the composition of matter while going through the lyophilization process.

Therapeutic Endpoints and Dosages

One of ordinary skill will appreciate that, from a medical practitioner's or patient's perspective, virtually any alleviation or prevention of an undesirable symptom associated with a cancerous condition (e.g., pain, sensitivity, weight loss, and the like) would be desirable. Additionally, any reduction in tumor mass or growth rate is desirable, as well as an improvement in the histopathological picture of the tumor. Thus, as used herein, the terms “treatment”, “therapeutic use”, or “medicinal use” used herein shall refer to any and all uses of the claimed compositions which remedy a disease state or symptoms, or otherwise prevent, hinder, retard, or reverse the progression of disease or other undesirable symptoms in any way whatsoever.

An effective dosage and treatment protocol may be determined by conventional means, starting with a low dose in laboratory animals and then increasing the dosage while monitoring the effects, and systematically varying the dosage regimen as well. Animal studies, preferably mammalian studies, are commonly used to determine the maximal tolerable dose, or MTD, of bioactive agent per kilogram weight. Those skilled in the art regularly extrapolate doses for efficacy and avoiding toxicity to other species, including human.

Before human studies of efficacy are undertaken, Phase I clinical studies in patients help establish safe doses. Numerous factors may be taken into consideration by a clinician when determining an optimal dosage for a given subject. Primary among these is the toxicity and half-life of the chosen heterologous gene product. Additional factors include the size of the patient, the age of the patient, the general condition of the patient, the particular cancerous disease being treated, the severity of the disease, the presence of other drugs in the patient, the in vivo activity of the gene product, and the like. The trial dosages would be chosen after consideration of the results of animal studies and the clinical literature.

Examples of the effective human doses of an adenoviral vector containing an H19 regulatory region or an IGF-2 P3 or P4 promoter region operatively linked to a heterologous gene encoding a cytotoxic agent are provided in U.S. Pat. No. 6,087,164.

For use in treating a cancerous condition in a subject, the present invention also provides in one of its aspects a kit or package, in the form of sterile-filled vials or ampoules, that contain the nucleic acid, the cationic polymer, the carbohydrate (e.g., trehalose) solution (i.e., the lyophilized composition). In one embodiment, the kit contains the polyplex solution prepared by the reverse method described herein in lyophilized form, in either unit dose or multi-dose amounts, wherein the package incorporates a label instructing reconstitution to form the reconstituted composition of matter and use of its contents for the treatment of cancer.

The invention having been described, the following examples are offered by way of illustration and not limitation.

### EXAMPLES

### Example 1: Current Polyplex Preparation Methods

In the current methods of polyplex preparation, the plasmid (BC-819) and cationic polymer (PEI) are supplied separately as two components. BC-819 (formerly known as DTA-H19) is provided in vials containing 5.3 mL at a concentration of 4 mg/mL, and polyethylenimine (PEI) is provided in vials containing 2.6 mL of 150 mM sterile solution.

TABLE 4
Preparation and Administration:

| Step A | Remove 1 BC-819 vial and 1 PEI vial from the freezer and let thaw for 15 minutes at room temperature. | | | |
|---|---|---|---|---|
| Step B | Handling the BC-819 | | Handling the PEI | |
| | 1. | Withdraw 15 mL from the sterile | 2. | Withdraw 17.6 mL from the sterile |
| | | dextrose 5% solution with a 20 cc | | dextrose 5% solution with a 20 cc |
| | | syringe and add to the empty 50 mL | | syringe and add to the empty 20 mL |
| | | glass bottle. | | glass bottle. |
| | 2. | Using a 5 cc syringe, withdraw 5 | 3. | Using a 5 cc syringe, withdraw 2.4 |
| | | mL of BC-819 and add to the 50 mL | | mL of PEI and add to the 20 mL |
| | | glass bottle with dextrose bringing | | bottle containing dextrose to a final |
| | | the final volume to 20 mL. | | volume of 20 mL. |

| | |
|---|---|
| Do not discard the used BC-819 vial. | Do not discard the used PEI vial. |

| | | |
|---|---|---|
| Step C | 1. | Transfer the contents of the smaller vial containing PEI using a 20 cc syringe |
| | | into the larger vial containing BC-819 and agitate for 10 seconds. |
| | | It is important to always work in this order (adding PEI to BC-819). |
| | 2. | Leave the mixture at room temperature for at least 15 minutes but for no more |
| | | than 1 hour (this step is important for PEI binding to BC-819). |
| | 3. | The final step is to bring the solution to a total of 50 mL with sterile dextrose 5% |
| | | which can be done by one of the following two methods: |

| | |
|---|---|
| a. | Using a 20 cc syringe, add 10 mL of sterile dextrose 5% to the vial |
| | containing the BC-819/PEI composition of matter to bring the total volume |
| | to 50 mL, OR |
| b. | Fill a 50 mL syringe with 10 mL of sterile dextrose 5% and pull the 40 mL |
| | mixture of BC-819/PEI into this syringe to make the total volume 50 mL. |

| | |
|---|---|
| Step D | The BC-819/PEI (50 mL) will be administered by intravesical instillation using a Foley catheter. The patients |
| | will be asked to hold the drug in the bladder for about 2 hours. |

### Example 2: Lyophilization Protocol

The purpose of this protocol is to prepare a ready to use lyophilized composition of matter containing BC-819 plasmid and in vivo-jetPEI® (polyethylenimine) In the protocol, the BC-819/in vivo-jetPEI® composition of matter is actually prepared on bed side of the patient.

Importantly, any deviation from the protocol preparation might result in a poor composition of matter quality that may affect the treatment results.

The use of this lyophilized composition of matter will reduce the risks of deviation in the treatment quality.

Materials and Equipment:

Materials

TABLE 5
DESCRIPTION

BC-819 solution
in vivo-jetPEI ® solution
5% sterile Trehalose solution
70% Ethanol
Latex Gloves
5 mL sterile pipettes
10 mL Sterile Pipettes
15 ml polypropylene tubes
125 ml polypropylene cups
Plastic beaker
Glassware + adaptator for lyophilizer
Desiccators + desiccant

Equipment

TABLE 6
DESCRIPTION

Magnetic stirrer
Magnet covering 50% of the bottom of the
125 ml polypropylene cup
Vortex
Freezer
Lyophilizer
Timer
Pipet Aid
Laminar flow hood

Safety Requirements

Latex or nitrile gloves and a clean lab coat should be worn while performing this procedure. Some materials requiring testing may be potentially biohazardous and should be disposed of appropriately.

Procedure:

All the solutions prepared for use in cell culture in vitro should be prepared in a laminar flow hood in aseptic conditions.
1. Preparations
   a. Thaw one vial of BC-819 and one vial of in vivo-jetPEI®.
   b. Mix 2.5 ml of BC-819 with 10 ml 5% trehalose solution in a 50 ml polypropylene tube.
   c. Mix 1.2 ml of in vivo-jetPEI® with 11.3 ml 5% trehalose solution in a 125 ml polypropylene cup and add a stirrer.
   d. Place the cup containing the in vivo-jetPEI®/Trehalose solution on the magnetic plate and power on.
   e. Drop the BC-819/trehalose solution into the in vivo-jetPEI®/trehalose solution at the rate of 2 ml/min.
   f. Let the composition of matter form for 3 additional min on the magnetic plate.
   g. Transfer the solution into a glassware fitted for lyophilization.
   h. Transfer the formed composition of matter in a −20 C±5 C freezer for 48 h.
   i. Transfer the bottles into a plastic container filled with ice.
   j. Connect the bottle to the lyophilizer, and power on for 72 h refreshing the ice.
   k. The powder obtained is kept in desiccators till reconstitution.
2. Reconstitute the powder by adding 25 ml of sterile double distilled water (DDW). The reconstituted solution should not show any precipitate. The appearance is slightly opalescent. The reconstituted solution is checked for potency assay. (See FIGS. 1A and 1B).

### Example 3: The Development of Stabilized BC819 Plasmid-Polyethylenimine Compositions of Matter

Materials:

BC-819 was prepared as previously described (see Ohana et al., International Journal of Cancer 98(5) (2002) 645-650; Ohana et al., The Journal of Gene Medicine 7(3) (2005) 366-374), and produced in large quantities at Altheas facilities (San Diego, USA). The in vivo-jetPEI® was purchased from Polyplus (Strasbourg, France).

100 ml glass vials, bromobutyl stoppers and sealers were purchases at Schott (Germany).

Methods:

Composition of Matter Preparation in a Small Scale:

5 ml of a 4 mg/ml BC-819 solution in TE buffer was added to 20 ml of a 5% trehalose solution.

2.4 ml of in vivo-jetPEI® was added to 22.6 ml of 5% trehalose solution. The plasmid solution was then added to the PEI solution while stirring to allow full homogenization of both components. The solution was incubated at room temperature for less than 1 hour. This way, 50 nil of a solution 0.4 mg/ml DNA complexed with in vivo-jetPEI® at the ratio N/P=6 (molar ratio of LPEI nitrogen (N) to DNA phosphate (P) was obtained. The indicated polyplex concentration refers to the plasmid DNA concentration of the sample.

Composition of Matter Preparation in a Medium Scale:

100 ml of a 4 mg/ml BC-819 solution in TE buffer was added to 400 ml of a 5% trehalose solution. 48 ml of in vivo-jetPEI® was added to 452 ml of 5% trehalose solution. The plasmid solution was then added to the PEI solution while stirring to allow full homogenization of both components. The solution was incubated at room temperature for less than 1 hour. This way, 1000 ml of a solution 0.4 mg/ml DNA complexed with in vivo-jetPEI® at the ratio

N/P=6 (molar ratio of LPEI nitrogen (N) to DNA phosphate (P) was obtained. The indicated polyplex concentration, refer to the plasmid DNA concentration of the sample.

The polyplex solution is filled in vials and is freeze dried as described below.

The solution is prepared in reverse order compared to the standard protocol. This reverse polyplex preparation protocol does not require any apparatus development and can be easily scaled up in any industrial facilities.

Lyophilization:

Pre-lyophilized composition of matter solution was freshly prepared as described above and transferred in 100 ml vials for freeze drying process.

The bottles samples went through a freeze drying cycle in a laboratory freeze dryer LyoBeta 25, including a freezing step at −45 C during at least 4 hours, a primary drying of at least 41 hours at 25 C, and a secondary drying of at least 8 hours both drying at pressure of 0.150 mb.

Particle Size Determination:

The z-average particle diameter of the samples was measured using a zetasizer (nano-s) from Malvern instruments (Herrenberg, Germany), angle 180° at a wavelength of 633 mm at 25° C. (viscosity, refractive index)

Zeta Potential Determination:

The zeta potential of the samples was determined using the zetasizer (nano-s) from Malvern instruments (Herrenberg, Germany).

Spectrophotometry/Turbidity:

A nanodrop (ThermoScientific nanodrop2000 spectophotometer) was used to perform the test: the samples were checked at 260 nm to evaluate the DNA concentration in the solution and at 600 nm to evaluate the turbidity of the solution.

The spectra of the solution was run between 200 and 400 nm. (See FIG. 5).

Electrophoresis Gel:

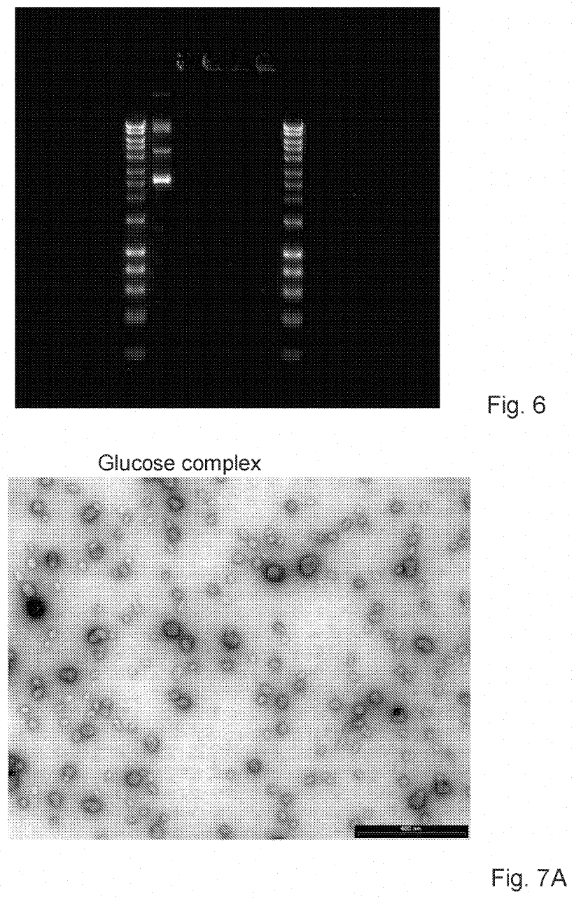

The samples were loaded on a 1% agarose gel and run for 1 h at 100 mV. This test recognizes any DNA plasmid that is not complexed with in vivo-jetPEI®, or any degradation. (See FIG. 6).

TEM (Shape and Size or Atomic Force Microscopy):

Samples were adsorbed to Formvar coated copper grids. Grids were stained with 1% (w/v) uranyl acetate and air-dried. Samples were viewed with Tecnai 12 TEM 100 kV (Phillips, Eindhoven, the Netherlands) equipped with MegaView II CCD camera and Analysis® version

3.0 software (SoftImaging System GmbH, Münstar, Germany).

The samples were checked in order to evaluate the shape of the polyplexcs in solution or in the dried product.

pH of the Solution:

The pH of the solutions was checked using a pHmeter (mettler Toledo, Swiss).

Osmometry:

The osmometry of the samples was tested using a Fiske® micro-osmometer model 201 (Advanced Instruments, Inc. Norwood, Mass.).

Transfection:

Water Content/LOD:

In Vivo Potency Evaluation:

Stability:

Results:

The samples dissolved immediately upon rehydration with filtered double distilled water (DDW) in the amount of their original volume and incubated for 5 min prior to use in the following tests.

TABLE 7
Particle size determination:

| Solutions | size (d · nm) avg |
|---|---|
| Method of the prior art (in glucose 5%) | 45.6 |
| Prelyophilization solution (reverse preparation | 35.7 |
| in trehalose) 0.4 mg/ml | |
| Lyophilized powder after reconstitution 0.4 mg/ml | 49.01 |
| Lyophilized powder after reconstitution 0.8 mg/ml | 264.9 |

As shown in FIG. 3, the composition of matter obtained after lyophilization show no significant difference with a fresh composition of matter prepared with trehalose or with glucose solutions. When freshly prepared, the glucose solution showed a rage of size from 50 to 240 nm.

TABLE 8
zeta potential determination

| Solutions | zeta potential (mV) |
|---|---|
| Method of the prior art (in glucose 5%) | 113 |
| Prelyophilization solution (reverse preparation in | 110 |
| trehalose) 0.4 mg/ml | |
| Lyophilized powder after reconstitution 0.4 mg/ml | 116 |
| Lyophilized powder after reconstitution 0.8 mg/ml | 125 |

Spectrophotometry:

As shown in Table 9 below and in FIG. 5, the DNA concentration in the rehydrated solution shows no significant difference when compared to fresh prepared solution in trehalose or glucose.

TABLE 9

| Sample ID | Nucleic Acid Conc. | Unit | A260 | A280 | 260/280 | 260/230 | Type | Factor |
|---|---|---|---|---|---|---|---|---|
| 1 | 483.2 | ng/μl | 9.663 | 6.105 | 1.58 | 1.75 | DNA | 50 |
| 1 | 485.1 | ng/μl | 9.703 | 6.145 | 1.58 | 1.75 | DNA | 50 |
| 1 | 483.5 | ng/μl | 9.67 | 6.13 | 1.58 | 1.75 | DNA | 50 |
| 2 | 472.3 | ng/μl | 9.446 | 5.757 | 1.64 | 1.88 | DNA | 50 |
| 2 | 472.2 | ng/μl | 9.444 | 5.73 | 1.65 | 1.88 | DNA | 50 |
| 2 | 473.3 | ng/μl | 9.465 | 5.751 | 1.65 | 1.88 | DNA | 50 |
| 3 | 446.4 | ng/μl | 8.929 | 5.561 | 1.61 | 1.81 | DNA | 50 |
| 3 | 446.6 | ng/μl | 8.933 | 5.558 | 1.61 | 1.81 | DNA | 50 |
| 3 | 447.7 | ng/μl | 8.954 | 5.568 | 1.61 | 1.81 | DNA | 50 |

1: method of the prior art (in glucose 5%).
2: prelyophilized solution (reverse preparation in trehalose) 0.4 mg/ml.
3: lyophilized powder after reconstitution 0.4 mg/ml

TEM (Shape and Size or Atomic Force Microscopy):

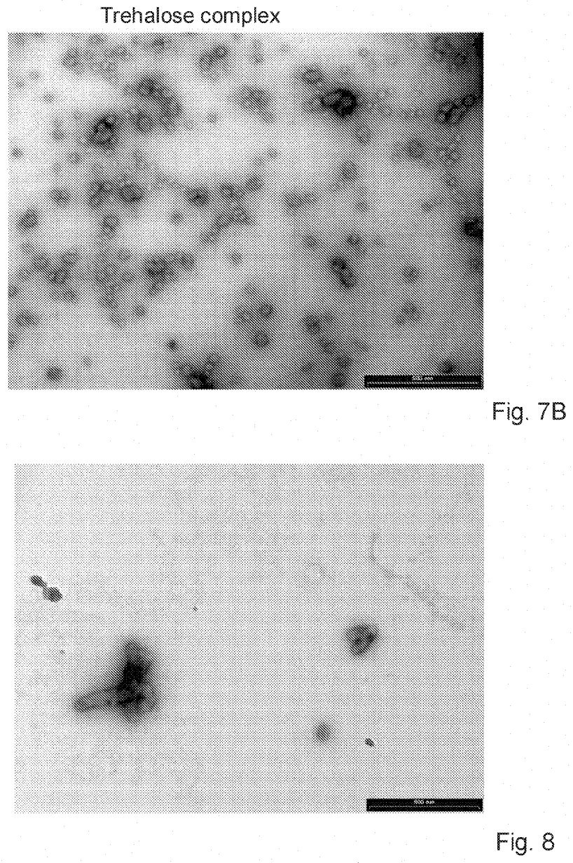

The samples observed show no difference when prepared in glucose or trehalose. (See FIGS. 7A-B). FIG. 8 shows the sample after precipitation.

pH of the Solution:

All the samples prepared in 5% glucose or in trehalose before or after lyophilization were around pH 3.0.

Osmometry:

TABLE 10

| Solutions | Osmometer (mosm/kg) |
|---|---|
| 5% glucose solution | 274 |
| 5% trehalose solution | 147 |
| method of the prior art (in glucose 5%) | 240 |
| Method of the prior art in trehalose 5% | 135 |
| Prelyophilization solution (reverse preparation | 138 |
| in trehalose) 0.4mg/ml | |

Transfection:

Water Content/LOD:

The water content tested in the dried product after the first cycle was 2.4%.

In Vivo Potency Evaluation:

Stability:

Conclusions:

In conclusion, the results of this study demonstrate that BC-819/in vivo-jetPEI® polyplex at relatively high concentration can be prepared in a large scale using a technique that can be easily transferred to an industrial scale.

The quality of the composition of matter produced allows the lyophilization of the BC-819/in vivo-jetPEI® without the need of additional excipients.

The preliminary results show that the lyophilized polyplex keeps the needed characterization of the composition of matter prepare at bed side with additional advantages: the dried product kept for 2 weeks at 5 C showed repeatable size of the polyplex, repeatable zeta potential, and stable product after rehydration.

Thus, the composition of matter can be provided to clinical study sites as a lyophilized powder ready for reconstitution. This novel formulation will ensure reproducible administrations for clinical use.

### Example 4: Examination of Stability of Different Solutions

Experiments were completed to examine the stability of different solutions at specific, pre-determined time intervals (i.e., within 1 hour from preparation/reconstitution, 3 hours, 3 days, 1 week). Specifically, three complex solutions (in glucose 5% (prior art preparation), in trehalose freshly prepared (prelyophilization (reverse preparation method)), and in trehalose reconstituted from lyophilizate (reconstituted in 10 nil IV water for injection) were examined and compared for the following parameters: nanosizer, zeta potential, osmometer, pH meter, transmission electron microscopy (TEM) with negative stain, nanodrop-DNA concentration, turbidity at 600 nm, and gel electrophoresis.

In addition to these tests, the following tests will be performed on the dried product: scanning electron microscopy (SEM), atomic force microscopy (AFM), Karl Fisher, Cryo-TEM, and x-Ray.

Appearance

The prior art preparation (glucose sample) turned yellow after 3 hours at room temperature, while the prelyophilization (reverse process in trehalose) and the reconstituted lyophilized product showed no changes in appearance.

TEM

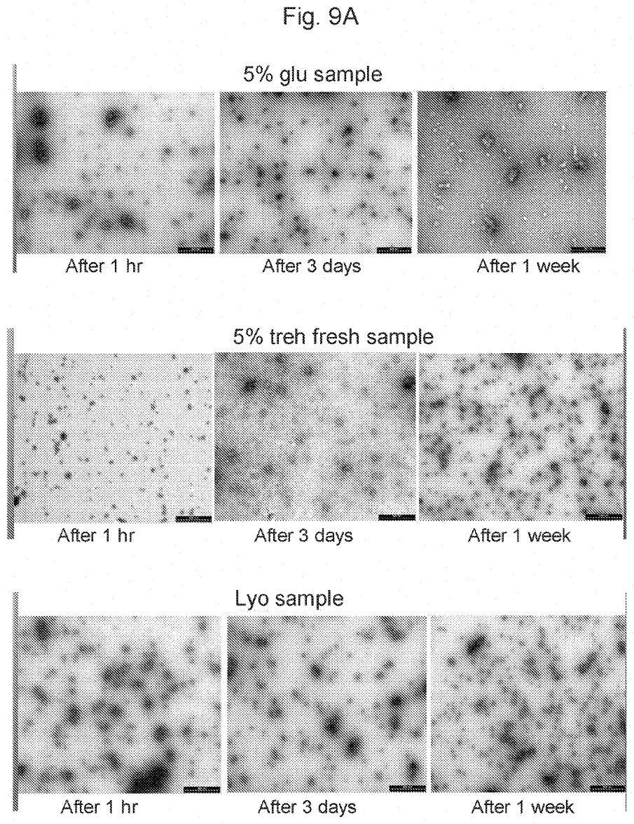

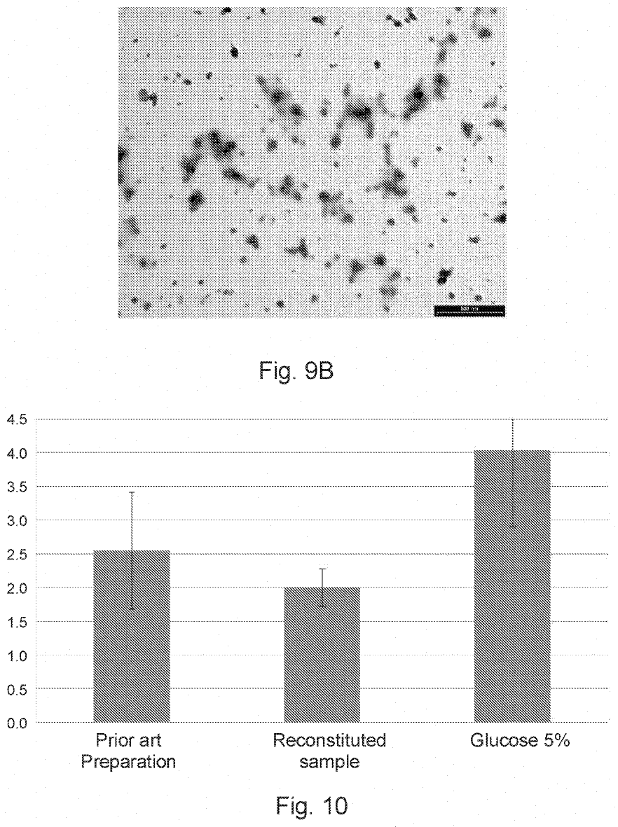

The results of transmission electron microscopy (TEM) are shown in FIG. 9A. The three pictures on the first line show the complex prepared according to the standard preparation (small dark spots) at three different time points. After one week, fewer dark spots are seen, which can indicate the complex degradation.

The three pictures in the second line show the pre-lyophilized sample (reverse preparation) at three time points. No decrease in the dark spots is observed.

Finally, the three pictures in the bottom line show the reconstituted product at three time points. Again, no decrease in the dark spots is observed.

FIG. 9B shows the precipitate complex in trehalose

pH Meter

Table 11 below shows the range of pH values observed:

TABLE 11

| | Time | | | |
|---|---|---|---|---|
| Sample | 1 hr | 3-5 hr | 3 days | 1 week |
| After reconstitution | 2.94-2.96 | 2.93-2.97 | 2.97-3.00 | 2.92-3.03 |
| (lyo IV) | | | | |
| Prelyophilization | 2.9-2.99 | 2.88-3.01 | 2.85-3.03 | 2.96-3.00 |
| (treh iv) | | | | |
| Prior art preparation | 2.86-2.98 | 2.84-2.99 | 2.86-2.93 | 2.83-2.9 |
| (glu bag) | | | | |

These results show that there is no change in pH between the solutions and the various time intervals.

Osmolarity (Mosm/Kg)

Osmolarity is measured to assess the concentration of solid particles from a liquid. Table 12 below shows the range of values observed.

TABLE 12

Time

| Sample | 1 hr | 3-5 hr | 3 days | 1 week |
|---|---|---|---|---|
| After reconstitution | 99-120 | 102-122 | 118-120 | 97-125 |
| (lyo IV) | | | | |
| Prelyophilization | 132-134 | 130-137 | 129-163 | 134-165 |
| (treh iv) | | | | |
| Prior art preparation | 243-247 | 241-250 | 241-247 | 239-253 |
| (glu bag) | | | | |

TABLE 13

| Solutions | Glu 5% | Treh 5% |
|---|---|---|
| Osmometer (mosm/l) | 280 | 135 |
| Mw(gr/mol) | 180.16 | 378.33 |

The results above show that no difference is observed between the fresh solution and the lyophilizate in trehalose. The complex in glucose shows higher osmolarity. Experiments will be performed to examine and understand the implication of this difference when administered to the bladder.

Nanosize (d.nm)

The DLS method takes into consideration the viscosity and the refractive index of the components to set the particle size. Table 14 below shows the range of values (nm) observed.

TABLE 14

| | Time | | | |
|---|---|---|---|---|
| Sample | 1 hr | 3-5 hr | 3 days | 1 week |
| After | 81.25-90.3 | 74.42-91.75 | 87.28-91.95 | 92.19-105.3 |
| reconstitution | | | | |
| (lyo IV) | | | | |
| Prelyophiliza- | 41.39-51.58 | 42.69-51 | 42.94-54.46 | 48.93-55.47 |
| tion (treh iv) | | | | |
| Prior art | 51.29-94 | 48.78-90.33 | 58.71-74.03 | 57.79-68.92 |
| preparation | | | | |
| (glu bag) | | | | |

These results show that there is no significant change over time in the particle size for each solution.

Zeta Potential (mV)

The zeta potential is the electrokinetic potential in colloidal dispersions and is an indicator of the stability of system. Table 15 below shows the range of values observed.

TABLE 15

| | Time | | | |
|---|---|---|---|---|
| Sample | 1 hr | 3-5 hr | 3 days | 1 week |
| After reconstitution | 64.9-66.9 | 61.9-67.2 | 64.6-67.1 | 64.3-66.7 |
| (lyo IV) | | | | |
| Prelyophilization | 59.7-68 | 59-66.9 | 61.4-63.4 | 63.8-65.2 |
| (treh iv) | | | | |
| Prior art preparation | 68.4-71.4 | 66.6-70.8 | 65.2-66.9 | 66.3-69.6 |
| (glu bag) | | | | |

TABLE 16

| Zeta potential (mV) | Stability behavior of the colloid |
|---|---|
| From 0 to ±5 | Rapid coagulation or flocculation |
| From ±10 to ±30 | Incipient instability |
| From ±30 to ±40 | Moderate stability |
| From ±40 to ±60 | Good stability |
| More than ±61 | Excellent stability |

These results show that the solutions exhibited stability over the time periods measured and that there is no difference between all of the samples checked.

DNA Concentration (ng/μl)

Table 16 below shows the range of values observed.

TABLE 16

| | Time | | | |
|---|---|---|---|---|
| Sample | 1 hr | 3-5 hr | 3 days | 1 week |
| After | 455-459.4 | 438.4-441.1 | 424.5-457.4 | 435.6-440.1 |
| reconstitution | | | | |
| (lyo IV) | | | | |
| Prelyophiliza- | 455.7-466.6 | 459-464.1 | 458.1-467.8 | 452.1-463.6 |
| tion (treh iv) | | | | |
| Prior art | 456.6-477.4 | 461.8-475.6 | 442-507.4 | 481.3-491.5 |
| preparation | | | | |
| (glu bag) | | | | |

These results show that the DNA concentration remains in the correct range over time. No decrease was observed.

Turbidity (cell/ml)

Table 17 below shows the range of values observed (abs at 600 nm).

TABLE 17

| | Time | | | |
|---|---|---|---|---|
| Sample | 1 hr | 3-5 hr | 3 days | 1 week |
| After | 0.01-0.014 | 0.009-0.012 | 0.009-0.012 | 0.011-0.014 |
| reconstitution | | | | |
| (lyo IV) | | | | |
| Prelyophiliza- | 0.004-0.009 | 0.004-0.009 | 0.006-0.008 | 0.004-0.007 |
| tion (treh iv) | | | | |
| Prior art | 0.005-0.01 | 0.009-0.016 | 0.007-0.012 | 0.009-0.014 |
| preparation | | | | |
| (glu bag) | | | | |

No turbidity in solutions over time was measurable. Additional experiments will be performed to measure turbidity.

Spectra from 250 to 600 nm

Over the time observed, the complex prepared in glucose showed absorbance at around 380 nm (violet). Additional experiments will be performed to determine what the residual compound is that caused this absorbance.

### Example 5: In Vivo Testing

Two million HCT-116 cells were injected into the back of athymic nude mice. When the tumors reached the size to be treated, the mice received three injections of prior art or reconstituted sample vs. glucose 5% (control). The results are shown in FIG. 10.

1.-92. (canceled)

93. A composition comprising
at least one nucleic acid,
at least one cationic polymer, and
a carbohydrate solution comprising at least one carbohydrate,
wherein the nucleic acid and the cationic polymer form a complex, and
wherein the w/w ratio of the at least one carbohydrate to the nucleic acid-cationic polymer complex is between 50 and 1,000.

94. The composition according to claim 93, being in a lyophilized form.

95. The composition according to claim 93, wherein said complex is in the form of a nanoparticle comprising the at least one nucleic acid and at least one cationic polymer.

96. The composition according to claim 95, wherein the nanoparticle is between about 40 nm and about 90 nm in size.

97. The composition according to claim 93, wherein the ratio is below 500.

98. The composition according to claim 93, wherein the ratio is below 250.

99. The composition according to claim 93, wherein the nucleic acid is DNA or RNA or a base analog of DNA or RNA.

100. The composition according to claim 93, wherein the nucleic acid is selected from the group consisting of DNA, shRNA, siRNA, and an oligonucleotide.

101. The composition according to claim 93, wherein the nucleic acid comprises a plasmid comprising an H19 regulatory sequence.

102. The composition according to claim 93, wherein at least one carbohydrate is a compound having the general formula (CH2O)n and selected from saccharides, polysaccharides and oligosaccharides and is not mannitol and is not sorbitol.

103. The composition according to claim 93, wherein the at least one carbohydrate is selected from trehalose, glucose, sucrose, lactose, mannitol, sorbitol, raffinose, PVP, and dextrose.

104. The composition according to claim 93, wherein the at least one carbohydrate is a carbohydrate other than glucose and other than sucrose.

105. The composition according to claim 93, wherein the at least one carbohydrate is trehalose.

106. The composition according to claim 93, wherein the at least one cationic polymer is a hydrophilic cationic polymer.

107. The composition according to claim 93, wherein the at least one cationic polymer is polyethylenimine (PEI), optionally linear PEI.

108. The composition according to claim 106, wherein the mole ratio of amine groups of the PEI to the moles of the phosphate groups of the nucleic acid is between 2 and 10.

109. A composition according to claim 93, the composition being prepared by a preparation process comprising adding a nucleic acid/carbohydrate solution into a cationic polymer/carbohydrate solution, under conditions permitting formation of a complex between the at least one nucleic acid and the at least one cationic polymer.

110. The composition according to claim 109, wherein the preparation process comprises:
(a) obtaining a solution comprising at least one nucleic acid and at least one carbohydrate;
(b) obtaining a solution comprising at least one cationic polymer and at least one carbohydrate;
(c) adding the nucleic acid/carbohydrate solution into the cationic polymer/carbohydrate solution, under conditions causing formation of a complex between the at least one nucleic acid and the at least one cationic polymer; and
(d) optionally lyophilizing the combined nucleic acid/cationic polymer/carbohydrate solution to form the lyophilized composition.

111. The composition according to claim 93, being in a pre-lyophilized form.

112. The composition according to claim 93, wherein the at least one cationic polymer is an amphiphilic cationic polymer.
